SEQUENCE LISTING (9 sequences)

SEQ ID NO: 1 - DNA, 3575 bp, Homo sapiens
  misc_feature: Incyte ID No  28115.15
gcggccgccgcacgccgcgccgccagcatgacggccgggaggcaggcgagcgcgcgcagcccgtcggccaggcacaggtcgagcagcaggggctgtggggtcaccttctcttcctctcttctgccaccccaagtcgtctgtgttgcgtgtcatgcagtgggacagcacgttggtccggacctcaagctacccacgttctagtcccgcctctgtcatttcctagagctgtggcctcggatccctccctggtgccacagcagctgagtgcgcatcaaatctgaagaaaatctacacagtacagacagtacagatattttggagtgtatacaataatatccctcctgtgactagcctgcctttgagatgtagttatcatttaatgagaggagagaggcgaccactttgggaagaggagagtaatgcaaagggtggcgtatggaagatgaaagtccccaaggacagcacgtccacagtttggaaagagttgctgttagcaaccatcggggaacagttcacagactgtgccgcagcagatgatgaagtaataggagttagtgtcagtgttcgggaccgagaagacgtcgtccaagtctggaatgtaaatgcctctttagtgggtgaagcgactgttttagaaaagatctatgaacttctgccccacataacttttaaagcagtattttataaaccccatgaagagcatcatgcttttgaaggtggacgtggaaaacactaattgcactctgtaaagaattctttgtcctttgctgattggttttggaaacggtcttaacaggagggagagtgaagagaagacttgccgaagccgatgctggtcaatttagagtgggtttctgtccttgcagatgggcaattaggactcaaagtggcaggtggggtggggggagattgtgtgggtttctaccgtcacattacttgctttttaaaaaaaacacacacaaaaaactttttttttggctttctaaattctctgttcttttcactctgattttttttttttttattggccgtttaaaattccttattcagcctaattattgttttctacactcccctttcattgaggcacaagaaatgggtttccctttaagtagctctgctgtacctagttaatgagaatatgcataatcgtgacaatactgcttttgaataccacactgtacaaggaggaacactagcttggagaaatctgtcttctgattatttgttgtgacacattcctatgtactgtgagctgggcattgtttttttctgtcccagggaaaagaaaaaccatacttaatgtgattttgcactgacagcacacaaatctttaattttcctttttaaaacatttttttttagtgataaaaggaaaataatagttggattgcataacatgaaaactataatcacaattcagtatcaggatataaatcaagctctaagtctttggaccctctgtggaaacatttctaacccgaattttaatttggccttttcaaccaaaggcttagagtggtagcaagggatttcttccaataggaaacctgatgtttctgatttaaagagaaggtgatattttaattgtttgaattaagctccttgcaaagttcgggtgtgtttacccacttagggctttctttcccagtcaacgctaacacattttgttaaatgatccctttccctgctcacattgtgtgtcatttctcatcatcagtagtcctccagcctgggcagctgtccccaccctttttcatgtaggtgcaggaagttaaatctcatttccaggatgcatgtgaacatttacaaagttgaactttgagtgcattctgctcatatgaattattgggattgttgatatatattgtattatgctaccaaagaaatattggttttattagaaggaaatggtcatcctctggaccatggagactagctcagaatacgctgatttccctctcctgactttgccaagcctttggctgcttttgcctgataaagggcagggccatctgaagacacttcccccagtcggctttggagtcacgggagctagtgcctgctcacacatttttcaaaagggcagtgcactcagaacttcactgtacctgggatttttaattcctcttgcagtgttgaccagcagagagacttgaggctactttaagcctccactatgtgtttgtagataaaattctccattcaaacattttaaaggactttgaacattatctgcttatggaagttgtgcccttcacttggttagtaaccacctcagccataatacttaccatcataggtttcttaaaatgctttttttttttccctaaacttgagtttccttagtgatttcaaaatgaagtataagaatatcagatccagttagcaaaagcctaggacttgtttctccaaacattgtactaacattcaacttgttttaaaattatgactcaagaattttaaaaaattattctggacatgaattaaaacttttttataatataagtatttttctgattgaaaaaaggatataattgacttcactctaattgtcatgtatatttccataagtaaatggattttgaagtatttttattttttgaactttatttaaagcatttgtgatgacatgttcaacttttgcatgtatgtagcctttgaagtaaaaataaataggaatgttaggctcacgttaatatttctagtttctgtttatgatcatggaacatgactagcacacaaagtagatttttgccccagtgaacttctgcaactgtttctgaccctttagaaaataccaactgtgagaaggatttaatttgcctaatgttaaaaacctatcatctaaattactttcagagaggattttgtcagattttggtttggaaattgttttaattgaatatttaatcataatagactttgtatgaactaagctgatttaatcagtttaaaaacctctcattaccttcttagattaaagaagctaatactgttttgagatagaaggaaatattttgtggggcatgtgacttttgtgttaaggcagtttacccatcttggctgatgtggggaaagtgcaaacttccagtagaatcccggtcttttcatccaagtgcttgcaggggcagccttgtagtctgcaaagaaccatgcagaaaccaaagagcttgcttttttgtctttgtaattccttagggtctgtgatatatgccaggaaatctgattgaaactaatatgctttttattctcccattattccctaaattgttatatcacaggcacttgcctacattgggaaagtaaggacaaataatacccatttaaagtaacttgcctatattttgactgtctttcgtaagcacttgctgaactgctgacttaactcagttttaagatttcctctttccccccaacttttccacatttcttaaatgagggtgtaatttactgcaaacatgtacatttaatataatattgctgttagattcgtggtacactttatgaagcaaaaacatgaaaaggaaaccacaactctaataaactttaaattaaagattaagatgactctt

SEQ ID NO: 2 - DNA, 575 bp, Homo sapiens
  misc_feature: Incyte ID No 29997.1
ttcggctcgagcaagtggaaccactggcttggtggattttgctagatttttctgatttttaaactcctgaaaaatatcccagataactgtcatgaagctggtaactatcttcctgctggtgaccatcagcctttgtagttactctgctactgccttcctcatcaacaaagtgccccttcctgttgacaagttggcacctttacctctggacaacattcttccctttatggatccattaaagcttcttctgaaaactctgggcatttctgttgagcaccttgtggaggggctaaggaagtgtgtaaatgagctgggaccagaggcttctgaagctgtgaagaaactgctggtaaccacagcttgggaggctaatctgccaaagggaggcgctatcacacttggtgtgacatcaagataaagagcggaggtggatggggatggaagatgatgctcctatcctccctgcctgaaacctgttctaccaattatagatcaaatgccctaaaatgtagtgacccgtgaaaaggacaaataaagcaatgaatacatttaaactcagaccatcgaatggaaaa

SEQ ID NO: 3 - DNA, 693 bp, Homo sapiens
  misc_feature: Incyte ID No 197927.7
cttggcagagagcgccctgggaaatgcggatatagaaggtcaggagaacatctctcctggttatacagcattccaggtgggctttcatgtggttgaggataatatcattagcaccaaaggtgaaaatccgtggaagctcatcttatttcagcagaagagtaggatgggccttgtggcacgagtaagccaaacaagggaaaggcattcaccatcaattgactcctcatctgtttttaagagggaaatctgagttttcaaggaaagccgaatacagttgccaagttgccagtcaaagaaacaatgtcaacacctgctcatagagatggaattcctaacccggaatattgcccttgaattacaacgagaaaaagcacctcttcaagtgagatggcagaccacctcaggatggaaatgtatttcttcatccccctctctacttccctgtgacctcctgcaaataagggagagacaaagagaagagcagaagaacagaagaatggagtggcaaagagaagagaaggagtgactgaacattgacaagactttggttggggatggtcagagaggagattagctgctagacggccacactgcggagaaagatcattttcccactccatcccccttccagctccccatccatctcaccaagagccagctccaccactcaataaaaccttacattcatctttc

SEQ ID NO: 4 - DNA, 1171 bp, Homo sapiens
  misc_feature: Incyte ID No 221807.2
gattcccataaagcacatggtctaatctgttacgtaacagcaagacagcgtcacctcacctgttctcgccctcaaatgggaacgctggcctgggactaaagcatagaccaccaggctgagtatcctgacctgagtcatccccagggatcaggagcctccagcagggaaccttccattatattcttcaagcaacttacagctgcaccgacagttgcgatgaaagttctaatctcttccctcctcctgttgctgccactaatgctgatgtccatggtctctagcagcctgaatccaggggtcgccagaggccacagggaccgaggccaggcttctaggagatggctccaggaaggcggccaagaatgtgagtgcaaagattggttcctgagagccccgagaagaaaattcatgacagtgtctgggctgccaaagaagcagtgcccctgtgatcatttcaagggcaatgtgaagaaaacaagacaccaaaggcaccacagaaagccaaacaagcattccagagcctgccagcaatttctcaaacaatgtcagctaagaagctttgctctgcctttgtaggagctctgagcgcccactcttccaattaaacattctcagccaagaagacagtgagcacacctaccagacactcttcttctcccacctcactctcccactgtacccacccctaaatcattccagtgctctcaaaaagcatgtttttcaagatcattttgtttgttgctctctctagtgtcttcttctctcgtcagtcttagcctgtgccctccccttacccaggcttaggcttaattacctgaaagattccaggaaactgtagcttcctagctagtgtcatttaaccttaaatgcaatcaggaaagtagcaaacagaagtcaataaatatttttaaatgtcacagatcaaaattgtttccttcaaatggggtctgccaattcacaaccagatgacccattttaccctattcactgcagactgaatccagattctacacatacttatccccaccaagaccctcactctgtctccattggcctacttgttcatctttcactcattcgacaaatctttttgaggtaagagcgaggtgggacaaaaaaaaaaagcataccaatgaaccagacccggtcttattaaagataatataggttt

SEQ ID NO: 5 - DNA, 484 bp, Homo sapiens
  misc_feature: Incyte ID No 236582.3
gagaggccaccgggacttcagtgtctcctccatcccaggagcgcagtggccactatggggtctgggctgccccttgtcctcctcttgaccctccttggcagctcacatggaacagggccgggtatgactttgcaactgaagctgaaggagtcttttctgacaagttcctcctatgagtccagcttcctggaattgcttgaaaagctctgcctcctcctccatctcccttcagggaccagcgtcaccctccaccatgcaagatctcaacaccatgttgtctgcaacacatgacagccattgaagcctgtgtccttcttggcccgggcttttgggccggggatgcaggaggcaggccccgaccctgtctttcagcaggcccccaccctcctgagtggcaataaataaaattcggtatgctgaaaaataaaaaaaagcccaaaaaaaaaacccaaaccaacaagaaacaaaacaaaaaaggggcccc

SEQ ID NO: 6 - DNA, 561 bp, Homo sapiens
  misc_feature: Incyte ID No 242745.1
gctttctcaggagcgcgggcgaggccggcgctggaggggcgaggaccgggtataagaagcctcgtggccttgcccgggcagccgcaggttccccgcgcgccccgagcccccgcgccatgaagctcgccgccctcctggggctctgcgtggccctgtcctgcagctccgctgctgctttcttagtgggctcggccaagcctgtggcccagcctgtcgctgcgctggagtcggcggcggaggccggggccgggaccctggccaaccccctcggcaccctcaacccgctgaagctcctgctgagcagcctgggcatccccgtgaaccacctcatagagggctcccagaagtgtgtggctgagctgggtccccaggccgtgggggccgtgaaggccctgaaggccctgctgggggccctgacagtgtttggctgagccgagactggagcatctacacctgaggacaagacgctgcccacccgcgagggctgaaaaccccgccgcggggaggaccgtccatccccttcccccggcccctctcaataaacgtggttaagagcaaaaa

SEQ ID NO: 7 - DNA, 1116 bp, Homo sapiens
  misc_feature: Incyte ID No 257520.9
aaggaggactcggtccactccgttacgtgtacatccaacaagatcggcgttaaggttctttttcttcaggatcaggcacagagactgactgaatggctccaattatcaggatttgaaaacccagtatcagaatctaccactttgtatggaggaggggaggagacgagtggggaccagcttgacatccagtcttcacctggacatatggaaagaacaaatgtgcgatctgctcgttccctctgaaggtctctgttacgtatttcctcctctcctccagagcataataaccaatgactgctctcagaaagaacaggcctgagggagagggaaaagcggatacccacctgtgtcgctgtttgcgtgccaagtccaggaacagtccatacagccctgctgcatcccacgacgctgtcacaaagcaggagttcatccgaggccaagatgttaattattcatactgcatgactgaggattttggaggcagagagagattcatctgcaatatttggaacaccaatggaggtctacgtcaacacagaatttatacagcagctggtgctagtcagagctaatgacagaatttcagtttaataaaaagacccccaactgagcacaccatcttgaaaaaagtatacttatcaaacagctttcaatcagttcaagagagacaccttaattggggagaggaagaattgcagagtagtttgtaatcatgccaattccagatcaataactgcatgtctgttctttggtagaaatagcttttgctttatattaagtaatcacatatatattctctctatttggataaggaaaccttcgctttatttgacaatgtataatgatatactcttctaattcacctctgtgtcttcacaataaacatgagtaaaatttagacaagtgatggtaaaggtcaatataattatttatttttaaaataaattttgtatctaacaggaaagcagttcttatgaaatttttatattttcaaaaattgttttgttcaaataaaattttatgagtaaagttaaatgatggtggtttaattataagaagagtatactgaacactgataatctgatttgtatgcaattacttcggggaaggctgtac

SEQ ID NO: 8 - DNA, 865 bp, Homo sapiens
  misc_feature: Incyte ID No 1093420.1
tctaagttgacaaggcagcaacttcagttcatttgttcaagtgtactttgatttgcatagtttcataacctgttagcatctgctcattgataattctgagcttgtgctttcctgacgattttttactgcagaggtggaaaagtactaaacggcaatttgtttctttaattttcctgtgcaaacaaagtcagggaacaaagaaagatgcatggtgactgcaaaatatcattattattttatcaacaatgataatacaatactccatgagtacaacttcttagtaataaatgcacataaaagcaagatagccaggaagtgcaaaggacaaattcccataatgctacatgaattgttttgcatagggcccactgtagcctcctgctgcctaatagataaattgaaaattttgcaactctacatctatctcaccagctttcagactgtgcactagctatgccaaattgactaatgtggatcaagactccatctcagtaaataaatagatagataaaaagcgctgcagtagctgtggcctcaccctgaagtcagcgggcccaggcctacctcactctctcccttggcagagaagcagacgtccatagctcctctccctcacaagcgctcccagcctgccctccagctgctgctctcccctcccagtctctactcactgggatgaggttaggtcatgaggacaccaaaaacctaaaaataaacaaaaagccaaacaagccttagcttttcttaaagactgaaatgcctggaagtgtccctttatttataaaataacttttgtcatatttcttatacatgtttcttgtaagaaattcagaaactacagacaaagagagtggaaattacccac

SEQ ID NO: 9 - DNA, 909 bp, Homo sapiens
  misc_feature: Incyte ID No 1384445.1
gggggtcagttttgttactctgagagctgttcacttctctgaattcacctagagtggttggaccatcagatgtttgggcaaaactgaaagctctttgcaaccacacaccttccctgagcttacatcactgcccttttgagcagaaagtctaaattccttccaagacagtagaattccatcccagtaccaaagccagataggccccctaggaaactgaggtaagagcagtctctaaaaactacccacagcagcattggtgcaggggaacttggccattaggttattatttgagaggaaagtcctcacatcaatagtacatatgaaagtgacctccaaggggattggtgaatactcataaggatcttcaggctgaacagactatgtctggggaaagaacggattatgccccattaaataacaagttgtgttcaagagtcagagcagtgagctcagaggcccttctcactgagacagcaacatttaaaccaaaccagaggaagtatttgtggaactcactgcctcagtttgggtaaaggatgagcagacaagtcaactaaagaaaaaagaaaagcaaggaggagggttgagcaatctagagcatggagtttgttaagtgctctctggatttgagttgaagagcatccatttgagttgaaggccacagggcacaatgagctctcccttctaccaccagaaagtccctggtcaggtctcaggtagtgcggtgtggctcagctgggtttttaattagcgcattctctatccaacatttaattgtttgaaagcctccatatagttagattgtgctttgtaattttgttgttgttgctctatcttattgtatatgcattgagtattaacctgaatgttttgttacttaaatattaaaaacactgttatcctacagtt

(19) United States
(12) Patent Application Publication
(10) Pub. No.: US 2003/0124580 A1
(43) Pub. Date: Jul. 3, 2003

(54) Lung surfactant molecules

(75) Inventors: Michael G. Walker, Sunnyvale, CA (US); Peter A. Spiro, Berkeley, CA (US); Lynn E. Murry, Fayetteville, AR (US)

Correspondence Address: INCYTE GENOMICS, INC., 3160 PORTER DRIVE, PALO ALTO, CA, 94304, US

(73) Assignee: Incyte Genomics, Inc., Palo Alto, CA

(21) Appl. No.: 10/237,435

(22) Filed: Sep. 6, 2002

Related U.S. Application Data
(60) Provisional application No. 60/317,822, filed on Sep. 7, 2001.

(51) Int. Cl. C12Q 1/68; C07H 21/04; C12P 21/02; C12N 5/06; C12N 9/64

(52) U.S. Cl. 435/6; 435/69.1; 435/226; 435/320.1; 435/325; 536/23.2

(57) ABSTRACT

The invention provides cDNAs that are significantly co-expressed with known lung surfactant and surfactant synthesis genes. The invention also provides compositions, expression vectors, host cells, proteins encoded by the cDNAs and antibodies which specifically bind the proteins. The invention also provides methods for the diagnosis, prognosis, treatment and evaluation of therapies for lung disorders.

This application claims benefit of provisional application Serial No. 60/317,822, filed Sep. 7, 2001.

### FIELD OF THE INVENTION

The invention relates to a combination comprising a plurality of isolated cDNAs, and their encoded proteins, that are co-expressed with known lung surfactant molecules specifically in lung tissue that are useful in the diagnosis, prognosis, treatment and evaluation of therapies for and treatment of lung disorders.

### BACKGROUND OF THE INVENTION

Lung surfactant is a surface-active lipoprotein complex at the air-liquid interface in the lung. Surfactant plays two primary roles. Its first role is in respiration where surfactant stabilizes alveoli and airways during expiration, improves bronchial clearance, and regulates airway liquid balance (Hohlfeld et al. (1997) Eur Respir J 10:482-91; Griese (1999) Eur Respir J 13:1455-76; and Banerjee et al. (2000) J Biomater Appl 15:140-59). Its second role is to protect against damage to the lung caused by inhaled particles or pathogens where surfactant mediates phagocytosis, regulates lymphocyte proliferation, suppresses cytokine secretion and transcription factor activation, and protects against cell damage from extracellular proteases (Hohlfeld, supra; Wright (1997) Physiol Rev 77:931-62; and Griese, supra). The production and function of surfactant molecules is affected both by cancer (Shijubo et al. (1995) Eur Respir J 8:403-6; Wiedmann (2000) Control Release 65:43-7) and by radiotherapy or chemotherapy used to treat cancer.

Surfactant contains a variety of proteins. Surfactant proteins A1 (SFTPA1) and A2 (SFTPA2) facilitate reduction of surface tension by surfactant phospholipids, aid in phospholipid synthesis, secretion and recycling, and, along with surfactant protein D (SFPTD), play a major role in defending the lung against pathogens, in clearing particulates, and in immunomodulation (Wright, supra; Kumar and Snyder (1998) Indian J Pediatr 65:629-41; Vaandrager and van Golde (2000) Biol Neonate 77:9-13; Awasthi et al. (2001) Am J Respir Crit Care Med 163:389-97; Crouch and Wright (2001) Annu Rev Physiol 63:521-54; Ofek et al. (2001) Infect Immun 69:24-33; and Pryhuber et al. (1996) Am J Physiol 270:L714-21). Surfactant proteins B (SFTPB) and C (SFTPC) are essential for adsorption of surfactant lipids to the air-liquid interface (Bachurski et al. (1995) J Biol Chem 270:19402-7; Vorbroker et al. (1995) Am J Physiol 268:647-56; and Robertson et al. (2000) Mol Med Today 6:119-24).

Genes that participate in surfactant synthesis include thyroid transcription factor 1 (TTF-1), which regulates transcription of respiratory epithelium-specific genes including SFT PA1, SFTPA2, SFTPB, SFTPC (Yan and Whitsett (1997) J Biol Chem 272:17327-32; Whitsett and Glasser (1998) Biochim Biophys Acta 1408:303-11), and the sodium-dependent phosphate transporter (NaPi3B); which regulates the uptake of phosphate necessary for surfactant synthesis (Traebert et al. (1999) Am J Physiol 277:L868-73). Most surfactant proteins are produced in a pro-form and are activated by proteolytic cleavage carried out by proteases produced in the lung. The surfactant-associated proteases include the aspartic protease, napsin A, which is expressed in normal alveolar epithelium and is a current target of pharmaceutical research (Rosse et al. (2000) J Comb Chem 2:461-6; Schauer-Vukasinovic et al. (2001) Biochim Biophys Acta 1524:51-6); and pepsin C, which is expressed in mucal secretory surfaces. Lung epithelial cells produce secretory leukocyte protease inhibitor (SLPI; van Wetering et al. (2000) J Investig Med 48:359-66) as protection against damage by the extracellular activity of proteases. SLPI is the primary defense of the conducting airways against neutrophil elastase (Jaumann et al. (2000) Munich Lung Transplant Group Eur Respir J 15:1052-7).

The role of lung surfactants in human lung disease was recently reviewed by Griese (supra) who wrote, “Biochemical surfactant abnormalities of varying degrees have been described in obstructive lung diseases (asthma, bronchiolitis, chronic obstructive pulmonary disease, and following lung transplantation), infectious and suppurative lung diseases (cystic fibrosis, pneumonia, and human immunodeficiency virus), adult respiratory distress syndrome, pulmonary oedema, other diseases specific to infants (chronic lung disease of prematurity, and surfactant protein-B deficiency), interstitial lung diseases (sarcoidosis, idiopathic pulmonary fibrosis, and hypersensitivity pneumonitis), pulmonary alveolar proteinosis following cardiopulmonary bypass, and in smokers.” Although respiratory distress syndrome and meconium aspiration in infants are treated with surfactants and surfactant replacement therapy is on the horizon for some pulmonary conditions, the role of surfactant in the pathophysiology of the majority of lung disorders is still unknown (Griese, supra).

The present invention satisfies a need in the art by providing a plurality of expressed cDNAs, and their encoded proteins, each of which may be used alone or as part of a combination in the diagnosis, prognosis, treatment and evaluation of therapies for and treatment of lung disorders.

### SUMMARY OF THE INVENTION

The invention provides a combination comprising a plurality of cDNAs having the nucleic acid sequences of SEQ ID NOs:1-9 and the complements thereof and a cDNA encoding pepsin C that are co-expressed with one or more known lung surfactant genes in a plurality of biological samples.

The invention also provides an isolated cDNA having a nucleic acid sequence selected from SEQ ID NOs:1-9 and the complements thereof. In different aspects, each cDNA is used as a probe, in an expression vector, and as a diagnostic in assessing the prognosis and treatment of a lung disorder. The invention also provides a composition comprising a cDNA or the complement thereof and a labeling moiety.

The invention further provides a method of using the combination or a cDNA of the combination to screen a plurality of molecules to identify at least one ligand which specifically binds a cDNA of the combination, the method comprising contacting the combination or a cDNA from the combination with a sample under conditions to allow specific binding; and detecting specific binding, thereby identifying a ligand which specifically binds the cDNA. In one embodiment, the molecules are selected from DNA molecules, RNA molecules, peptides and proteins. In another embodiment, a biological sample is screened, and the identified protein is a transcription factor.

The invention additionally provides a method for using the combination or a cDNA of the combination to detect differential expression in a sample containing nucleic acids, the method comprising hybridizing the combination or the cDNA to the nucleic acids under conditions for formation of one or more hybridization complexes; detecting hybridization complex formation, comparing complex formation with standards wherein the comparison indicates differential expression in the sample. In one embodiment, the cDNAs of the combination are immobilized on a substrate. In a second embodiment, the sample is from lung. In a third embodiment, expression when compared to standards is diagnostic of lung disorders. In a fourth embodiment, the lung disorder is lung cancer.

The invention provides a purified protein encoded by a cDNA of the invention. The invention also provides a method for using the protein to screen a plurality of molecules to identify and purify a ligand which specifically binds the protein. In one embodiment, the molecules to be screened are selected from agonists, antagonists, antibodies, DNA molecules, RNA molecules, peptides, and proteins.

The invention additionally provides a method for detecting differential expression of a protein in a sample, the method comprising quantifying the amount of a protein in a sample, and comparing the amount with standards, thereby detecting differential expression. In one embodiment, the sample is from lung. In a second embodiment, differential expression is diagnostic of a lung disorder. In a third embodiment, the lung disorder is lung cancer.

The invention further provides a method of using a protein to prepare and purify an antibody which specifically binds the protein comprising immunizing an animal with the protein or peptide under conditions to elicit an antibody response; isolating animal antibodies; attaching the protein to a substrate; contacting the substrate with isolated antibodies under conditions to allow specific binding; and dissociating antibody from protein, thereby obtaining purified antibody.

The invention provides an antibody that specifically binds a protein of the invention. The invention also provides methods for using the antibody to detect differential expression of a protein in a sample, the method comprising combining the antibody with a sample under conditions for specific binding, detecting antibody:protein complex formation, comparing complex formation with standards, thereby detecting differential expression of the protein in the sample. In one embodiment, the sample is from lung. In another embodiment, differential expression is diagnostic of a lung disorder. In a third embodiment, the lung disorder is lung cancer. The invention additionally provides a composition comprising a cDNA, a protein or an antibody that specifically binds a protein and a pharmaceutical carrier for use in treating a lung disorder.

### DESCRIPTION OF THE TABLE

Table 1 shows the differential expression of the cDNAs in lung tumor. The first column shows SEQ ID NO; the second column, the microarray (GEM) used for the experiments, the third column, the log 2 (Cy5/Cy3) value; the fourth column, the description of the normal lung sample; the fifth column, the description of the lung tumor sample; and the sixth column, the donor ID. Abbreviations include NSC=non-small cell, SCC=squamous cell carcinoma; CA=carcinoma (or cancer), HG=HumanGenome GEM, and UG=UNIGEM. Shading below indicates those experiments in which the same tissue was arrayed twice. The duplicated experiments are grouped by Donor ID.

### DESCRIPTION OF THE INVENTION

It must be noted that as used herein and in the appended claims, the singular forms “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment, and an antibody-peptide fusion protein.

“Antigenic determinant” refers to an antigenic or immunogenic epitope, structural feature, or region of an oligopeptide, peptide, or protein which is capable of inducing formation of an antibody that specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody is of diagnostic or therapeutic interest. The arrangement of at least two and up to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

A “bispecific molecule” has two different binding specificities and can be bound to two different molecules or two different sites on a molecule concurrently. Similarly, a “multispecific molecule” can bind to multiple (more than two) distinct targets, one of which is a molecule on the surface of an immune cell. Antibodies can perform as or be a part of bispecific or multispecific molecules.

Cancer includes an adenocarcinoma, squamous cell carcinoma, small cell carcinoma, non-small cell carcinoma, endobronchial, neuroendocrine or spindle cell carcinoid, and caseating granuloma.

A “combination” comprises at least two cDNAs selected from the group consisting of SEQ ID NOs:1-9 as presented in the Sequence Listing and a cDNA encoding pepsin C and the complements of these cDNAs.

The “complement” of a cDNA of the Sequence Listing refers to a nucleic acid molecule which is completely complementary over the full length of the nucleic acid sequence.

“cDNA” refers to an isolated polynucleotide, nucleic acid molecule, or any fragment thereof that contains from about 400 to about 12,000 nucleotides. It may have originated recombinantly or synthetically, may be double-stranded or single-stranded, may represent coding and noncoding 3′ or 5′ sequence, and generally lacks introns.

The phrase “cDNA encoding a protein” refers to a nucleic acid whose sequence closely aligns with sequences that encode conserved regions, motifs or domains identified by employing analyses well known in the art. These analyses include BLAST (Basic Local Alignment Search Tool; Altschul (1993) J Mol Evol 36:290-300; Altschul et al. (1990) J Mol Biol 215:403-410) and BLAST2 (Altschul et al. (1997Nucleic Acids Res 25:3389-3402) which provide identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

A “composition” refers to the polynucleotide and a labeling moiety; a purified protein and a pharmaceutical carrier or a heterologous, labeling or purification moiety; an antibody and a labeling moiety or pharmaceutical agent; and the like.

“Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a cDNA or a protein can also involve the replacement of a hydrogen by an acetyl, acyl, alkyl, amino, formyl, or morpholino group (for example, 5-methylcytosine). Derivative molecules retain the biological activities of the naturally occurring molecules but may confer longer lifespan or enhanced activity.

“Differential expression” refers to an increased or upregulated or a decreased or downregulated expression as detected by absence, presence, or at least two-fold change in the amount of transcribed messenger RNA or translated protein in a sample.

An “expression profile” is a representation of gene expression in a sample. A nucleic acid expression profile is produced using sequencing, hybridization, or amplification technologies and mRNAs (or cDNAs made from the mRNAs) from a sample. A protein expression profile, although time delayed, mirrors the nucleic acid expression profile and may use proteomic technologies or platforms, antibody or protein arrays, enzyme-linked immunosorbent assays, fluorescence-activated cell sorting, spatial immobilization such as 2D-PAGE and western analysis to detect protein expression in a sample. The nucleic acids, proteins, or antibodies may be used in solution or attached to a substrate, and their detection is based on methods and labeling moieties well known in the art. Expression profiles may also be evaluated by methods such as electronic northern analysis, guilt-by-association, and transcript imaging. Expression profiles may be used to compare gene expression in normal versus diseased tissues. The correspondence between mRNA and protein expression has been discussed by Zweiger (2001, Transducing the Genome. McGraw-Hill, San Francisco, Calif.) and Glavas et al. (2001; T cell activation upregulates cyclic nucleotide phosphodiesterases 8A1 and 7A3, Proc Natl Acad Sci 98:6319-6342) among others.

“Fragment” refers to a chain of consecutive nucleotides from about 50 to about 5000 base pairs in length. Fragments may be used in PCR or hybridization technologies to identify related nucleic acid molecules and in binding assays to screen for a ligand. Such ligands are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3′-T-C-A-G-5′. The degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul et al. (1997). BLAST2 may be used in a standardized and reproducible way to insert gaps in one of the sequences in order to optimize alignment and to achieve a more meaningful comparison between them. “Similarity” as applied to proteins uses the same algorithms but takes into account conservative substitutions of nucleotides or residues.

“Isolated” or “purified” refers to any molecule or compound that is separated from its natural environment and is from about 60% free to about 90% free from other components with which it is naturally associated.

“Labeling moiety” refers to any reporter molecule including radionuclides, enzymes, fluorescent, chemiluminescent, or chromogenic agents, substrates, cofactors, inhibitors, or magnetic particles than can be attached to or incorporated into a polynucleotide, protein, or antibody. Visible labels and dyes include but are not limited to anthocyanins, β glucuronidase, biotin, BIODIPY, Coomassie blue, Cy3 and Cy5, 4,6-diamidino-2-phenylindole (DAPI), digoxigenin, fluorescein, FITC gold, green fluorescent protein, lissamine, luciferase, phycoerythrin, rhodamine, spyro red, silver, streptavidin, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

A “lung surfactant gene” refers to a polynucleotide which has been previously identified as useful in the diagnosis, prognosis, treatment, and evaluation of therapies associated with lung disorders. Typically, this means that the known gene is differentially expressed at higher (or lower) levels in tissues from patients with a lung disorder when compared with normal expression in any tissue. The lung surfactant genes used in this invention are surfactant protein A1 (SFTPA1), surfactant protein A2 (SFTPA2), surfactant protein C (SFTPC), surfactant protein D (SFTPD), secretory leukocyte protease inhibitor (SLPI), thyroid transcription factor 1 (TIF1), sodium-dependent phosphate transporter (NaPi3B), and napsin A.

“Lung disorder” refers to any surfactant-associated lung condition or disease including cell damage induced by adult respiratory distress syndrome (ARDS), allergy, asthma, bronchitis, cancer chemotherapy, chronic obstructive pulmonary disease (COPD), cystic fibrosis, emphysema, idiopathic pulmonary fibrosis (IPF), interstitial pneumonia with collagen disease (IPCD), meconium aspiration pulmonary alveolar proteinosis (PAP), pneumonia, pneumonitis, idiopathic pulmonary fibrosis (IPF), idiopathic pulmonary oedema, radiotherapy, sarcoidosis, or smoking.

“Oligonucleotide” refers a single-stranded molecule from about 18 to about 60 nucleotides in length which may be used in hybridization or amplification technologies or in regulation of replication, transcription or translation. Equivalent terms are amplicon, amplimer, primer, and oligomer.

A “pharmaceutical agent” may be an antibody, an antisense molecule, a bispecific molecule, a multispecific molecule, a peptide, a protein, a radionuclide, a small drug molecule, a cytospecific or cytotoxic drug such as abrin, actinomyosin D, cisplatin, crotin, doxorubicin, 5-fluorouracil, methotrexate, ricin, vincristine, vinblastine,, or any combination of these elements.

“Portion” refers to any part of a protein used for any purpose which retains at least one biological or antigenic characteristic of a native protein, but especially, to an epitope for the screening of ligands or for the production of antibodies.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid in a sample. Where targets are single stranded, probes are complementary single strands. Probes can be labeled for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis or an antigenic determinant of the protein identified using Kyte-Doolittle algorithms of the PROTEAN program (DNASTAR, Madison Wis.). An “oligopeptide” is an amino acid sequence from about five residues to about 15 residues that is used as part of a fusion protein to produce an antibody.

“Sample” is used in its broadest sense as containing nucleic acids, proteins, and antibodies. A sample may comprise a bodily fluid such as ascites, blood, cerebrospinal fluid, lymph, semen, sputum, urine and the like; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue, a tissue biopsy, or a tissue print; buccal cells, skin, hair, a hair follicle; and the like.

“Specific binding” refers to a precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which polynucleotides, proteins, or antibodies are bound and includes magnetic or nonmagnetic beads, capillaries or other tubing, chips, fibers, filters, gels, membranes, plates, polymers, slides, wafers, and microparticles with a variety of surface forms including channels, columns, pins, pores, trenches, and wells.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a protein or the polynucleotides that encode it. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The present invention identifies a combination comprising a plurality of cDNAs that can serve as surrogate diagnostic markers for lung disorders, potential therapeutics, or targets for the identification of therapeutics for lung disorders. In particular, the method identifies cDNAs cloned from mRNA transcripts that are significantly co-expressed with at least one known lung surfactant gene and differentially expressed in lung.

The method disclosed below provides for the identification of cDNAs that are expressed in a plurality of libraries. The nucleic acid sequences originate from human cDNA libraries derived from a variety of sources or can be selected from expressed sequence tags (ESTs), assembled polynucleotides, coding regions, introns, 5′ untranslated regions, and 3′ untranslated regions.

The cDNA libraries used in the analysis can be obtained from any human tissue including, but not limited to, adrenal gland, biliary tract, bladder, blood cells, blood vessels, bone marrow, brain, bronchus, cartilage, chromaffin system, colon, connective tissue, cultured cells, embryonic stem cells, endocrine glands, epithelium, esophagus, fetus, ganglia, heart, hypothalamus, immune system, intestine, islets of Langerhans, kidney, larynx, liver, lung, lymph, muscles, neurons, ovary, pancreas, penis, peripheral nervous system, phagocytes, pituitary, placenta, pleura, prostate, salivary glands, seminal vesicles, skeleton, spleen, stomach, testis, thymus, tongue, ureter, and uterus.

In a preferred embodiment, the cDNAs are assembled from related sequences, such as sequence fragments derived from a single transcript. In that sequencing technology allows for the reading of about 300 to about 700 bases at a time and that the usual clone insert contains about 5000 bases, even the sequence within a single clone must be sequenced in fragments and assembled. Assembly of the sequenced fragments of a cDNA can be performed using software now well known in the art or using the algorithm disclosed in U.S. Ser. No. 9,276,534, filed March 25, 1999, incorporated herein by reference.

The cDNAs of the invention define an expression profile against which to compare the expression pattern of biopsied and/or in vitro treated tissue. Experimentally, differential expression of the cDNAs can be evaluated by methods including, but not limited to, differential display by spatial immobilization or by gel electrophoresis, genome mismatch scanning, representational discriminate analysis, clustering, transcript imaging and array technologies. These methods may be used alone or in combination; in the present case, the preferred method is presented below and confirmed by transcript imaging and microarray data.

Application of Guilt-by-Association (GBA) Analysis

Guilt-by-Association (GBA) analysis for co-expression patterns described by Walker et al. (1999; Genome Res 9:1198-203; incorporated herein by reference), has been used to identify a set of nine cDNAs and a cDNA encoding pepsin C that are co-expressed with known lung surfactant and surfactant synthesis genes. Some of these cDNAs were unknown or previously uncharacterized, and some were not known to be so strongly associated with lung function or particular disorders of the lung. These cDNAs have utility as surrogate markers for known lung surfactant or surfactant synthesis genes and may be used as diagnostics, prognostics, therapeutics or therapeutic targets, or to test the efficacy or toxicity of therapies for lung disorders. Further, a protein or peptide encoded by any of the cDNAs can be used as a diagnostic, as a potential therapeutic, as a target for the identification or development of therapeutics, or for producing antibodies which specifically bind the protein or peptide. Still further, antibodies so produced are useful in the diagnosis, prognosis, treatment or evaluation of therapies for or treatment of lung disorders.

The procedure for identifying cDNAs that exhibit a statistically significant co-expression pattern with known lung surfactant genes is as follows. First, the presence or absence of a gene sequence in a cDNA library is defined: a gene is present in a cDNA library when at least one cDNA fragment corresponding to that gene is detected in a cDNA sample taken from the library, and a gene is absent from a library when no corresponding cDNA fragment is detected in the sample.

Second, the significance of gene co-expression is evaluated using a probability method to measure a due-to-chance probability of the co-expression. The probability method can be the Fisher exact test, the chi-squared test, or the kappa test. These tests and examples of their applications are well known in the art and can be found in standard statistics texts (Agresti (1990) Categorical Data Analysis, John Wiley & Sons, New York N.Y.; Rice (1988) Mathematical Statistics and Data Analysis, Duxbury Press, Pacific Grove Calif.). A Bonferroni correction (Rice, supra, p. 38⁴) can also be applied in combination with one of the probability methods for correcting statistical results of one gene versus multiple other genes. In a preferred embodiment, the due-to-chance probability is measured by a Fisher exact test, and the threshold of the due-to-chance probability is set preferably to less than 0.001, more preferably to less than 0.00001.

To determine whether two genes, A and B, have similar co-expression patterns, occurrence data vectors can be generated as illustrated in the table below. The presence of a gene occurring at least once in a library is indicated by a one, and its absence from the library, by a zero.

| | Library 1 | Library 2 | Library 3 | . . . | Library N |
|---|---|---|---|---|---|
| Gene A | 1 | 1 | 0 | . . . | 0 |
| Gene B | 1 | 0 | 1 | . . . | 0 |

For a given pair of genes, the occurrence data in the table above can be summarized in a 2×2 contingency table. The contingency table (below) presents co-occurrence data for gene A and gene B in a total of 30 libraries. Both gene A and gene B occur 10 times in the libraries.

| | Gene A Present | Gene A Absent | Total |
|---|---|---|---|
| Gene B Present | 8 | 2 | 10 |
| Gene B Absent | 2 | 18 | 20 |
| Total | 10 | 20 | 30 |

The contingency table summarizes and presents: 1) the number of times gene A and B are both present in a library; 2) the number of times gene A and B are both absent in a library; 3) the number of times gene A is present, and gene B is absent; and 4) the number of times gene B is present, and gene A is absent. The upper left entry is the number of times the two genes co-occur in a library, and the middle right entry is the number of times neither gene occurs in a library. The off diagonal entries are the number of times one gene occurs, and the other does not. Both A and B are present eight times and absent 18 times. Gene A is present, and gene B is absent, two times; and gene B is present, and gene A is absent, two times. The probability (“p-value”) that the above association occurs due to chance as calculated using a Fisher exact test is 0.0003. Associations are generally considered significant if a p-value is less than 0.01 (Agresti, supra; Rice, supra).

This method of estimating the probability for co-expression of two genes makes several assumptions. The method assumes that the libraries are independent and are identically sampled. However, in practical situations, the selected cDNA libraries are not entirely independent, because more than one library may be obtained from a single subject or tissue. Nor are they entirely identically sampled, because different numbers of cDNAs may be sequenced from each library. The number of cDNAs sequenced typically ranges from 5,000 to 10,000 cDNAs per library. In addition, because a Fisher exact co-expression probability is calculated for each gene versus all other assembled genes that occur in at least five libraries, a Bonferroni correction for multiple statistical tests is used.

Expression Profiles Produced Using GBA, TI and Array Technologies

Using GBA, ten cDNAs that exhibit highly significant co-expression probability with known lung surfactant genes have been identified. The results presented in EXAMPLE IV demonstrate the utility of the method by showing the highly significant co-expression among the known lung surfactant genes. EXAMPLE V shows the highly significant co-expression between each of the cDNAs of the invention and the known lung surfactant genes. These highly significant associations are summarized in the table below.

| SEQ ID | p-value | Lung Surfactant | Disorder |
|---|---|---|---|
| 1 | 16 | SFTPC | asthma |
| 2 | 40 | SFTPA2 and SFTPC | ARDS |
| 3 | 11 | SFTPC | adenocarcinoma |
| 4 | 19 | SFTPC | squamous cell CA |
| 5 | 26 | Napsin A | adenocarcinoma |
| 6 | 22 | SFTPC | adenocarcinoma |
| 7 | 29 | SFTPC | adenocarcinoma |
| 8 | 22 | SFTPC | COPD |
| 9 | 33 | SFTPC | COPD |
| Pepsin C | 19 | SFTPD | IPF, IPCD, and PAP |

In one embodiment, the invention encompasses a combination comprising a plurality of cDNAs having the nucleic acid sequences of SEQ ID NOs: 1-9, a cDNA encoding pepsin C, and the complements thereof. These cDNAs have been shown by the methods of the present invention to have significant, specific, and differential expression in lung disorders. The invention also provides methods for using the composition to detect lung disorders.

The table below shows the specificity of expression for SEQ ID NOs:2-9 and pepsin C in lung libraries. The probability was calculated using algorithms presented in U.S. Ser. No. 10/113,234 filed March 28, 2000, incorporated herein by reference. The first column shows SEQ ID NO; the second column, the number of cDNA fragments associated with the SEQ ID NO; the third column, the number of lung libraries in which the cDNA was expressed compared to the total number of libraries in which it was expressed; and the fourth column, the probability that expression of the cDNA is lung specific. It must be noted that expression of pepsin C was higher in lung than in any tissue of the gastrointestinal system with which it is usually assumed to be associated.

| SEQ ID | cDNAs | Lung/Total | Probability |
|---|---|---|---|
| 2 | 155 | 32/37 | P = 9.7e−38 |
| 3 | 25 | 18/25 | P = 3.2e−05 |
| 4 | 85 | 28/52 | P = 7.4e−18 |
| 5 | 33 | 20/24 | P = 1.4e−22 |
| 6 | 123 | 24/44 | P = 4.2e−20 |
| 7 | 36 | 18/21 | P = 9.1e−21 |
| 8 | 994 | 63/72 | P = 5.4e−83 |
| 9 | 87 | 24/40 | P = 1.8e−21 |
| pepsin C | 260 | 23/63 | P = 4.1e−15 |

The significant co-expression of SEQ ID NOs:1-9 with surfactant genes known to be associated with particular lung disorders is corroborated by the exemplary transcript image for SEQ ID NO:7 presented in EXAMPLE VI, and by the microarray data for SEQ ID NOs:1-4 and 7-9 presented in TABLE 1.

TABLE 1 shows the differential expression of the cDNAs represented by SEQ ID NOs: 1-4 and 7-9 in lung tumor. SEQ ID NOs:1-4 and 7-9 were significantly downregulated and pepsin C was significantly upregulated in matched normal/tumor samples from a single patient. Differential expression was significant if it exceeded log 2=1.3. In some cases, the tissues were arrayed twice; the duplicated experiments are grouped by Donor ID and shaded in TABLE 1.

The microarray data summarized below demonstrates the highly significant change in expression for SEQ ID NOs:1-9 and pepsin C in lung tumors compared to matched normal tissues. The first column shows SEQ ID NO; the second column, the microarray (GEM); the third column, the number of hybridizations with matched normal/tumor lung tissue; the fourth column, the percent of the hybridizations which exceeded a log 2=1.3 (˜two-fold, significant differential expression), the fifth column, the number of duplicated experiments, and the sixth column, the number of significant duplicates.

| | | | | | Signif. |
|---|---|---|---|---|---|
| SEQ ID | GEM | # Hybs | % Significant | # Duplicates | Duplicates |
| 1 | HG2 | 13 | 62 | | |
| 2 | HG1 | 44 | 95 | 15 | 14/15 |
| 3 | HG2 | 34 | 94 | 7 | 7 |
| 4 | HG1 | 32 | 93 | 10 | 9/10 |
| 7 | HG1 | 41 | 90 | 14 | 12/14 |
| 8 | HG5 | 34 | 82 | 10 | 9/10 |
| 9 | HG5 | 30 | 83 | 9 | 8/9 |
| pepsin C | UG2 | 1 | 100 | | |

cDNAs and Their Uses

cDNAs can be prepared by a variety of synthetic or enzymatic methods well known in the art. cDNAs can be synthesized, in whole or in part, using chemical methods well known in the art (Caruthers et al. (1980) Nucleic Acids Symp Ser (7):215-233). Alternatively, cDNAs can be produced enzymatically or recombinantly, by in vitro or in vivo transcription.

Nucleotide analogs can be incorporated into cDNAs by methods well known in the art. The only requirement is that the incorporated analog must base pair with native purines or pyrimidines. For example, 2, 6-diaminopurine can substitute for adenine and form stronger bonds with thymidine than those between adenine and thymidine. A weaker pair is formed when hypoxanthine is substituted for guanine and base pairs with cytosine. Additionally, cDNAs can include nucleotides that have been derivatized chemically or enzymatically.

cDNAs can be synthesized on a substrate. Synthesis on the surface of a substrate may be accomplished using a chemical coupling procedure and a piezoelectric printing apparatus as described by Baldeschweiler et al. (PCT publication WO95/251116). Alternatively, the cDNAs can be synthesized on a substrate surface using a self-addressable electronic device that controls when reagents are added as described by Heller et al. (U.S. Pat. No. 5,605,662). cDNAs can be synthesized directly on a substrate by sequentially dispensing reagents for their synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

cDNAs can be immobilized on a substrate by covalent means such as by chemical bonding procedures or UV irradiation. In one method, a cDNA is bound to a glass surface which has been modified to contain epoxide or aldehyde groups. In another method, a cDNA is placed on a polylysine coated surface and UV cross-linked to it as described by Shalon et al. (WO95/35505). In yet another method, a cDNA is actively transported from a solution to a given position on a substrate by electrical means (Heller, supra). cDNAs do not have to be directly bound to the substrate, but rather can be bound to the substrate through a linker group. The linker groups are typically about 6 to 50 atoms long to provide exposure of the attached cDNA. Preferred linker groups include ethylene glycol oligomers, diamines, diacids and the like. Reactive groups on the substrate surface react with a terminal group of the linker to bind the linker to the substrate. The other terminus of the linker is then bound to the cDNA. Alternatively, polynucleotides, plasmids or cells can be arranged on a filter. In the latter case, cells are lysed, proteins and cellular components degraded, and the DNA is coupled to the filter by UV cross-linking.

Hybridization

The cDNAs or fragments or complements thereof may be used in various hybridization technologies. The cDNAs may be labeled using a variety of reporter molecules by either PCR, recombinant, or enzymatic techniques. For example, a commercially available vector containing the cDNA is transcribed in the presence of an appropriate polymerase, such as T7 or SP6 polymerase, and at least one labeled nucleotide. Commercial kits are available for labeling and cleanup of such cDNAs. Radioactive (Amersham Biosciences (APB), Piscataway N.J.), fluorescent (Qiagen-Operon, Alameda Calif.), and chemiluminescent labeling (Promega, Madison Wis.) are well known in the art.

A cDNA may represent the complete coding region of an mRNA or be designed or derived from unique regions of the mRNA or genomic molecule, an intron, a 3′ untranslated region, or from a conserved motif. The cDNA is at least 18 contiguous nucleotides in length and is usually single stranded. Such a cDNA may be used under hybridization conditions that allow binding only to an identical sequence, a naturally occurring molecule encoding the same protein, or an allelic variant. Discovery of related human and mammalian sequences may also be accomplished using a pool of degenerate cDNAs and appropriate hybridization conditions. Generally, a cDNA for use in Southern or northern hybridizations may be from about 400 to about 6000 nucleotides long. Such cDNAs have high binding specificity in solution-based or substrate-based hybridizations. An oligonucleotide, a fragment of the cDNA, may be used to detect a polynucleotide in a sample using PCR.

The stringency of hybridization is determined by G+C content of the cDNA, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5×saline sodium citrate (SSC) with 1% sodium dodecyl sulfate (SDS) at 60° C., that permit the formation of a hybridization complex between nucleic acid sequences that contain some mismatches. Subsequent washes are performed with buffers such as 0.2×SSC with 0.1% SDS at either 45° C. (medium stringency) or 65°-68° C. (high stringency). At high stringency, hybridization complexes will remain stable only where the nucleic acids are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4.6-4.9).

Dot-blot, slot-blot, low density and high density arrays are prepared and analyzed using methods known in the art. cDNAs from about 18 consecutive nucleotides to about 5000 consecutive nucleotides in length are contemplated by the invention and used in array technologies. The preferred number of cDNAs on an array is at least about 100,000, a more preferred number is at least about 40,000, an even more preferred number is at least about 10,000, and a most preferred number is at least about 600 to about 800. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; WO95/11995; WO95/35505; U.S. Pat. Nos. 5,605,662; and 5,958,342.)

Screening and Purification Assays Using cDNAs

A cDNA may be used to screen a library or a plurality of molecules or compounds for a ligand which specifically binds the cDNA. Ligands may be DNA molecules, RNA molecules, peptide nucleic acid molecules, peptides, proteins such as transcription factors, promoters, enhancers, repressors, and other proteins that regulate replication, transcription, or translation of the polynucleotide in the biological system. The assay involves combining the cDNA or a fragment thereof with the molecules or compounds under conditions that allow specific binding and detecting the bound cDNA to identify at least one ligand that specifically binds the cDNA.

In one embodiment, the cDNA may be incubated with a library of isolated and purified molecules or compounds and binding activity determined by methods such as a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay. Protein binding may be confirmed by raising antibodies against the protein and adding the antibodies to the gel-retardation assay where specific binding will cause a supershift in the assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

The cDNA may be used to purify a ligand from a sample. A method for using a cDNA to purify a ligand would involve combining the cDNA or a fragment thereof with a sample under conditions to allow specific binding, recovering the bound cDNA, and using an appropriate agent to separate the cDNA from the purified ligand.

Protein Production and Uses

The full length cDNAs or fragments thereof may be used to produce purified proteins or peptides using recombinant DNA technologies described herein and taught in Ausubel (supra; Units 16.1-16.62). One of the advantages of producing proteins by these procedures is the ability to obtain highly-enriched sources of the proteins thereby simplifying purification procedures.

The proteins may contain amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.) and algorithms included in RasMol software (University of Massachusetts, Amherst Mass.) may be used to help determine which and how many amino acid residues in a particular portion of the protein may be substituted, inserted, or deleted without abolishing biological or immunological activity.

Expression of Encoded Proteins

Expression of a particular cDNA may be accomplished by cloning the cDNA into a vector and transforming this vector into a host cell. The cloning vector used for the construction of cDNA libraries in the LIFESEQ databases (Incyte Genomics, Palo Alto Calif.) may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into competent E. coli cells. Induction of the isolated bacterial strain with isopropylthiogalactoside (IPTG) using standard methods will produce a fusion protein that contains an N terminal methionine, the first seven residues of β-galactosidase, about 15 residues of linker, and the protein encoded by the cDNA.

The cDNA may be shuttled into other vectors known to be useful for expression of protein in specific hosts. Oligonucleotides containing cloning sites and fragments of DNA sufficient to hybridize to stretches at both ends of the cDNA may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the protein and the purification domain may also be used to recover the protein.

Suitable host cells may include, but are not limited to, mammalian cells such as Chinese Hamster Ovary (CHO) and human 293 cells, insect cells such as Sf9 cells, plant cells such as Nicotiana tabacum, yeast cells such as Saccharomyces cerevisiae, and bacteria such as E. coli. For each of these cell systems, a useful vector may also include an origin of replication and one or two selectable markers to allow selection in bacteria as well as in a transformed eukaryotic host. Vectors for use in eukaryotic host cells may require the addition of 3′ poly(A) tail if the cDNA lacks poly(A).

Additionally, the vector may contain promoters or enhancers that increase gene expression. Many promoters are known and used in the art. Most promoters are host specific and exemplary promoters includes SV40 promoters for CHO cells; T7 promoters for bacterial hosts; viral promoters and enhancers for plant cells; and PGH promoters for yeast. Adenoviral vectors with the rous sarcoma virus enhancer or retroviral vectors with long terminal repeat promoters may be used to drive protein expression in mammalian cell lines. Once homogeneous cultures of recombinant cells are obtained, large quantities of secreted soluble protein may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, proteins or portions thereof may be produced manually, using solid-phase techniques (Stewart et al. (1969) Solid-Phase Peptide Synthesis, W H Freeman, San Francisco Calif.; Merrifield (1963) J Am Chem Soc 5:2149-2154), or using machines such as the 431A peptide synthesizer (Applied Biosystems (ABI), Foster City Calif.). Proteins produced by any of the above methods may be used as pharmaceutical compositions to treat disorders associated with null or inadequate expression of the genomic sequence.

Screening and Purification Assays Using Proteins

A protein or a portion thereof encoded by the cDNA may be used to screen a library or a plurality of molecules or compounds for a ligand with specific binding affinity or to purify a molecule or compound from a sample. The protein or portion thereof employed in such screening may be free in solution, affixed to an abiotic or biotic substrate, or located intracellularly. For example, viable or fixed prokaryotic host cells that are stably transformed with recombinant nucleic acids that have expressed and positioned a protein on their cell surface can be used in screening assays. The cells are screened against a library or a plurality of ligands and the specificity of binding or formation of complexes between the expressed protein and the ligand may be measured. The ligands may be agonists, antagonists, antibodies, DNA molecules, enhancers, small drug molecules, immunoglobulins, inhibitors, mimetics, peptide nucleic acid molecules, peptides, pharmaceutical agents, proteins, and regulatory proteins, repressors, RNA molecules, ribozymes, transcription factors, or any other test molecule or compound that specifically binds the protein. An exemplary assay involves combining the mammalian protein or a portion thereof with the molecules or compounds under conditions that allow specific binding and detecting the bound protein to identify at least one ligand that specifically binds the protein.

This invention also contemplates the use of competitive drug screening assays in which neutralizing antibodies capable of binding the protein specifically compete with a test compound capable of binding to the protein or oligopeptide or fragment thereof. One method for high throughput screening using very small assay volumes and very small amounts of test compound is described in U.S. Pat. No. 5,876,946. Molecules or compounds identified by screening may be used in a model system to evaluate their toxicity, diagnostic, or therapeutic potential.

The protein may be used to purify a ligand from a sample. A method for using a protein to purify a ligand would involve combining the protein or a portion thereof with a sample under conditions to allow specific binding, recovering the bound protein, and using an appropriate chaotropic agent to separate the protein from the purified ligand.

Production of Antibodies

A protein encoded by a cDNA of the invention may be used to produce specific antibodies. Antibodies may be produced using an oligopeptide or a portion of the protein with inherent immunological activity. Methods for producing antibodies include: 1) injecting an animal, usually goats, rabbits, or mice, with the protein, or an antigenically-effective portion or an oligopeptide thereof, to induce an immune response; 2) engineering hybridomas to produce monoclonal antibodies; 3) inducing in vivo production in the lymphocyte population; or 4) screening libraries of recombinant immunoglobulins. Recombinant immunoglobulins may be produced as taught in U.S. Pat. No. 4,816,567.

Antibodies produced using the proteins of the invention are useful for the diagnosis of prepathologic disorders as well as the diagnosis of chronic or acute diseases characterized by abnormalities in the expression, amount, or distribution of the protein. A variety of protocols for competitive binding or immunoradiometric assays using either polyclonal or monoclonal antibodies specific for proteins are well known in the art. Immunoassays typically involve the formation of complexes between a protein and its specific binding molecule or compound and the measurement of complex formation. Immunoassays may employ a two-site, monoclonal-based assay that utilizes monoclonal antibodies reactive to two noninterfering epitopes on a specific protein or a competitive binding assay (Pound (1998) Immunochemical Protocols, Humana Press, Totowa N.J.).

Immunoassay procedures may be used to quantify expression of the protein in cell cultures, in subjects with a particular disorder or in model animal systems under various conditions. Increased or decreased production of proteins as monitored by immunoassay may contribute to knowledge of the cellular activities associated with developmental pathways, engineered conditions or diseases, or treatment efficacy. The quantity of a given protein in a given tissue may be determined by performing immunoassays on freeze-thawed detergent extracts of biological samples and comparing the slope of the binding curves to binding curves generated by purified protein.

Arrays

The cDNAs, proteins and antibodies may be used for a variety of purposes. For example, the combination of the invention may be used on an array. The array, in turn, can be used in high-throughput methods for detecting the expression of an identical or related polynucleotide or protein in a sample, screening a plurality of molecules or compounds to identify a ligand, or diagnosing a lung disorder

When the cDNAs, proteins or antibodies of the invention are employed on an array, the they are arranged in an ordered fashion so that each molecule is present at a specified location. Because the molecules are at specified locations, the patterns of complex formation are detectable. This is useful to detect a unique profile or to capture and categorize a bound ligand.

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt et al. (2000) Nature Biotechnol 18:989-94).

Labeling of Molecules for Assay

A wide variety of reporter molecules and conjugation techniques are known by those skilled in the art and may be used in various CDNA, polynucleotide, protein, peptide or antibody assays. Synthesis of labeled molecules may be achieved using commercial kits for incorporation of a labeled nucleotide such as ³²P-dCTP, Cy3-dCTP or Cy5-dCTP or amino acid such as ³⁵S-methionine. Polynucleotides, cDNAs, proteins, or antibodies may be directly labeled with a reporter molecule by chemical conjugation to amines, thiols and other groups present in the molecules using reagents such as BIODIPY or FITC (Molecular Probes, Eugene Oreg.).

The proteins and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. Nos. 3,817,837; 3,850,752; 3,939,350; 3,996,345; 4,277,437; 4,275,149; and 4,366,241.

Diagnostics

The cDNAs, or fragments thereof, may be used to detect and quantify differential gene expression; absence, presence, or excess expression of mRNAs; or to monitor mRNA levels during therapeutic intervention. Disorders associated with lung disorders include cell damage caused by adult respiratory ARDS, allergy, asthma, bronchitis, cancer (including but not limited to an adenocarcinoma, squamous cell carcinoma, small cell carcinoma, non-small cell carcinoma and endobronchial, neuroendocrine or spindle cell carcinoid), caseating granuloma, chemotherapy, COPD, cystic fibrosis, emphysema, IPF, IPCD, PAP, pneumonia, pneumonitis, IPF, idiopathic pulmonary oedema, radiotherapy, sarcoidosis, or smoking. These cDNAs can also be utilized as markers of treatment efficacy against the disorders noted above and other disorders, conditions, and diseases over a period ranging from several days to months. The diagnostic assay may use hybridization or amplification technology to compare gene expression in a biological sample from a patient to standard samples in order to detect altered gene expression. Qualitative or quantitative methods for this comparison are well known in the art.

For example, the cDNA may be labeled by standard methods and added to a biological sample from a patient under conditions for hybridization complex formation. After an incubation period, the sample is washed and the amount of label (or signal) associated with hybridization complexes is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

Gene Expression Profiles

A gene expression profile comprises a plurality of cDNAs or proteins and a plurality of detectable complexes, wherein each complex is formed by hybridization or specific binding between the cDNA, protein, or antibody and suitable ligands in a sample or combinatorial library. The cDNAs, proteins, and antibodies of the invention are used as elements on an array to analyze gene expression profiles. In one embodiment, the array is used to monitor the progression of disease. Researchers or clinicians can catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to produce expression profiles associated with a particular condition, disorder or disease; or treatment of the condition, disorder or disease. Novel treatment regimens may be tested in these animal models using arrays to establish and then follow expression profiles over time. In addition, arrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Assays Using Antibodies

Antibodies directed against antigenic determinants of a protein encoded by a cDNA of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety.

Protocols for detecting and measuring protein expression using either polyclonal or monoclonal antibodies are well known in the art. Examples include, but are not limited to, western analysis, ELISA, RIA, FACS, and arrays. Such immunoassays typically involve the formation of complexes between the protein and its specific antibody and the measurement of such complexes. These assays are specifically described in Pound (supra).

Therapeutics

The cDNAs and fragments thereof can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of a normal sequence, reduction or loss of an endogenous target protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

In addition, expression of a particular protein can be regulated through the specific binding of a fragment of a cDNA to a genomic sequence or an mRNA which encodes the protein or directs its transcription or translation. The cDNA can be modified or derivatized to any RNA-like or DNA-like material including peptide nucleic acids, branched nucleic acids, and the like. These sequences can be produced biologically by transforming an appropriate host cell with a vector containing the sequence of interest.

Molecules which regulate the activity of the cDNA or encoded protein are useful as therapeutics for lung disorders. Such molecules include agonists which increase the expression or activity of the polynucleotide or encoded protein, respectively; or antagonists which decrease expression or activity of the polynucleotide or encoded protein, respectively. In one aspect, an antibody which specifically binds the protein may be used directly as an antagonist or indirectly as a delivery mechanism for bringing a pharmaceutical agent to cells or tissues which express the protein.

Additionally, any of the proteins, or their ligands, or complementary nucleic acid sequences may be administered as pharmaceutical compositions or in combination with other appropriate therapeutic agents. Selection of the appropriate agents for use in combination therapy may be made by one of ordinary skill in the art, according to conventional pharmaceutical principles. The combination of therapeutic agents may act synergistically to affect the treatment or prevention of the conditions and disorders associated with an immune response. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration used by doctors and pharmacists may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

Transgenic Animal Models

Transgenic rodents that overexpress or underexpress a gene of interest may be inbred and used to model human diseases or to test therapeutic or toxic agents. (See, e.g., U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Stem Cells and Their Use

SEQ ID NOs:1-9 can be useful in the differentiation of stem cells. Eukaryotic stem cells are able to differentiate into the multiple cell types of various tissues and organs and to play roles in embryogenesis and adult tissue regeneration (Gearhart (1998) Science 282:1061-1062; Watt and Hogan (2000) Science 287:1427-1430). Depending on their source and developmental stage, stem cells can be totipotent with the potential to create every cell type in an organism and to generate a new organism, pluripotent with the potential to give rise to most cell types and tissues, but not a whole organism; or multipotent cells with the potential to differentiate into a limited number of cell types. Stem cells can be transformed with cDNAs which can be transiently expressed or can be integrated within the cell as transgenes.

Embryonic stem (ES) cell lines are derived from the inner cell masses of human blastocysts and are pluripotent (Thomson et al. (1998) Science 282:1145-1147). They have normal karyotypes and express high levels of telomerase which prevents senescence and allows the cells to replicate indefinitely. ES cells produce derivatives that give rise to embryonic epidermal, mesodermal and endodermal cells. Embryonic germ (EG) cell lines, which are produced from primordial germ cells isolated from gonadal ridges and mesenteries, also show stem cell behavior (Shamblott et al. (1998) Proc Natl Acad Sci 95:13726-13731). EG cells have normal karyotypes and appear to be pluripotent.

Organ-specific adult stem cells differentiate into the cell types of the tissues from which they were isolated. They maintain their original tissues by replacing cells destroyed from disease or injury. Adult stem cells are multipotent and under proper stimulation can be used to generate cell types of various other tissues (Vogel (2000) Science 287:1418-1419). Hematopoietic stem cells from bone marrow provide not only blood and immune cells, but can also be induced to transdifferentiate to form brain, liver, heart, skeletal muscle and smooth muscle cells. Similarly mesenchymal stem cells can be used to produce bone marrow, cartilage, muscle cells, and some neuron-like cells, and stem cells from muscle have the ability to differentiate into muscle and blood cells (Jackson et al. (1999) Proc Natl Acad Sci 96:14482-14486). Neural stem cells, which produce neurons and glia, can also be induced to differentiate into heart, muscle, liver, intestine, and blood cells (Kuhn and Svendsen (1999) BioEssays 21:625-630); Clarke et al. (2000) Science 288:1660-1663; Gage (2000) Science 287:1433-1438; and Galli et al. (2000) Nature Neurosci 3:986-991).

Neural stem cells can be used to treat neurological disorders such as Alzheimer disease, Parkinson disease, and multiple sclerosis and to repair tissue damaged by strokes and spinal cord injuries. Hematopoietic stem cells can be used to restore immune function in immunodeficient subjects or to treat autoimmune disorders by replacing autoreactive immune cells with normal cells to treat diseases such as multiple sclerosis, scleroderma, rheumatoid arthritis, and systemic lupus erythematosus. Mesenchymal stem cells can be used to repair tendons or to regenerate cartilage to treat arthritis. Liver stem cells can be used to repair liver damage. Pancreatic stem cells can be used to replace islet cells to treat diabetes. Muscle stem cells can be used to regenerate muscle to treat muscular dystrophies. (See, e.g., Fontes and Thomson (1999) BMJ 319:1-3; Weissman (2000) Science 287:1442-1446; Marshall (2000) Science 287:1419-1421; Marmont (2000) Ann Rev Med 51:115-134.)

### EXAMPLES

It is to be understood that this invention is not limited to the particular devices, machines, materials and methods described. Although particular embodiments known at the time the invention was made are described, equivalent embodiments can be used to practice the invention. The described embodiments are provided to illustrate the invention and are not intended to limit the scope of the invention which is limited only by the appended claims.

I cDNA Library Construction

RNA was purchased from Clontech (Palo Alto Calif.) or isolated from lung tissues. Some tissues were homogenized and lysed in guanidinium isothiocyanate; others were homogenized and lysed in phenol or a suitable mixture of denaturants, such as TRIZOL reagent (Invitrogen). The resulting lysates were centrifuged over CsCl cushions or extracted with chloroform. RNA was precipitated from the lysates with either isopropanol or sodium acetate and ethanol, or by other routine methods. Phenol extraction and precipitation of RNA were repeated as necessary to increase RNA purity.

In some cases, RNA was treated with DNAse. For most libraries, poly(A+) RNA was isolated using oligo d(T)-coupled paramagnetic particles (Promega), OLIGOTEX latex particles (Qiagen, Valencia Calif.), or an OLIGOTEX MRNA purification kit (Qiagen). Alternatively, RNA was isolated directly from tissue lysates using RNA isolation kits such as the POLY(A)PURE mRNA purification kit; Ambion, Austin Tex.).

In some cases, Stratagene (La Jolla Calif.) was provided with RNA and constructed the cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art. (See, e.g., Ausubel, 1997, supra, units 5.1-6.6). Reverse transcription was initiated using oligo d(T) or random primers. Synthetic oligonucleotide adapters were ligated to double stranded cDNA, and the cDNA was digested with the appropriate restriction enzyme(s). For most libraries, the cDNA was size-selected (300-1000 bp) using SEPHACRYL S1000, SEPHAROSE CL2B, or SEPHAROSE CLAB column chromatography (Amersham Biosciences (APB), Piscataway N.J.) or preparative agarose gel electrophoresis. cDNAs were ligated into compatible restriction enzyme sites of the polylinker of pBLUESCRIPT plasmid (Stratagene), pSPORT1 plasmid (Invitrogen), or pINCY (Incyte Genomics). Recombinant plasmids were transformed into competent E. coli cells including XL1-BLUE, XL1-BLUEMRF, or SOLR (Stratagene) or DH5α, DH10B, or ElectroMAX DH10B (Invitrogen).

II Isolation, Sequencing and Analysis of cDNA Clones,

Plasmids were recovered from host cells by either in vivo excision using the UNIZAP vector system (Stratagene) or cell lysis. Plasmids were purified using one of the following kits or systems: a Magic or WIZARD Minipreps DNA purification system (Promega); an AGTC Miniprep purification kit (Edge Biosystems, Gaithersburg Md.); and QIAWELL 8 plasmid, QIAWELL 8 Plus plasmid, QIAWELL 8 Ultra Plasmid purification systems or the REAL Prep 96 plasmid kit (Qiagen). Following precipitation, plasmids were resuspended in 0.1 ml of distilled water and stored, with or without lyophilization, at 4 C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a Fluoroskan II fluorescence scanner (Labsystems Oy, Helsinki, Finland).

The cDNAs were prepared for sequencing using the CATALYST 800 preparation system (ABI) or the HYDRA microdispenser (Robbins Scientific) or MICROLAB 2200 system (Hamilton, Reno Nev.) systems in combination with the DNA ENGINE thermal cyclers (MJ Research, Watertown Mass.). The cDNAs were sequenced using the PRISM 373, 377 or 3700 sequencing systems (ABI) and standard ABI protocols, base calling software, and kits. In the alternative, cDNAs were sequenced using the MEGABACE 1000 DNA sequencing system (APB). In another alternative, the cDNAs were amplified and sequenced using the PRISM BIGDYE Terminator cycle sequencing ready reaction kit (ABI). In yet another alternative, cDNAs were sequenced using solutions and dyes from APB. Reading frames for the ESTs were determined using standard methods (reviewed in Ausubel, supra, unit 7.7).

The cDNA sequence fragments derived from cDNA, extension, and shotgun sequencing were assembled and analyzed using a combination of software programs which utilize algorithms well known to those skilled in the art (Meyers, supra, pp 856-853).

III Assembly of cDNAs and Characterization of Sequences

The sequences used for co-expression analysis were assembled from EST sequences, 5′ and 3′ long read sequences, and full length coding sequences.

The cDNAs of this application were compared with assembled consensus sequences or templates found in the LIFESEQ GOLD database (Incyte Genomics). Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms (Incyte Genomics) that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10−8. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. Nos. 08/812,290 and 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.).

The BLAST software suite, freely available sequence comparison algorithms (NCBI, Bethesda Md.), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap×drop-off: 50; Expect: 10; Word Size: 11; and Filter: on. Identity or similarity is measured over the entire length of a sequence or some smaller portion thereof. Brenner et al. (1998; Proc Natl Acad Sci 95:6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The cDNA and any encoded protein were further queried against public databases such as the GenBank rodent, mammalian, vertebrate, prokaryote, and eukaryote databases, SwissProt, BLOCKS, PRINTS, PFAM, and Prosite.

IV Co-expression of Known Lung Surfactant and Surfactant Synthesis Genes

The co-expression of known lung surfactant and surfactant synthesis genes with each other has been investigated using GBA as described in Walker et al. (1999; Genome Res 9:1198-203) and the LIFESEQ Gold database (Incyte Genomics). The following table shows the highly significant co-expression of known lung surfactant and surfactant synthesis genes with each other. The entries in the table indicate the probability (expressed as −log P) that the observed co-expression for each pair of genes is due to chance, as measured by the Fisher Exact Test. The highest p-value is marked in bold for each lung surfactant molecule.

| Gene ID | SFTPA1 | SFTPA2 | SFTPC | SFTPD | SLPI | TTF1 | NaPi3B |
|---|---|---|---|---|---|---|---|
| SFTPA1 | NA | | | | | | |
| SFTPA2 | 23 | NA | | | | | |
| SFTPC | 23 | 71 | NA | | | | |
| SFTPD | 13 | 37 | 38 | NA | | | |
| SLPI | 6 | 14 | 17 | 14 | NA | | |
| TTF1 | 9 | 21 | 24 | 13 | 7 | NA | |
| NaPi3B | 11 | 26 | 32 | 20 | 15 | 20 | NA |
| Napsin A | 13 | 38 | 39 | 31 | 15 | 13 | 21 |

V Co-expression of cDNAs with Known Surfactant Genes

The table below shows the co-expression of cDNAs with known lung surfactant and surfactant synthesis genes. The entries in the table indicate the probability (−log P) that the observed co-expression for each pair of genes is due to chance, as measured by the Fisher Exact Test. Although co-expression analysis has shown high expression levels of pepsin C in the digestive, respiratory, and genital systems, the specific link of pepsin C to lung surfactants, particularly SFTPD with a p-value=19, is reported here for the first time.

| Gene ID | SFTPA1 | SFTPA2 | SFTPC | SFTPD | SLPI | TTF1 | NaPi3B | Napsin A | Pepsin C |
|---|---|---|---|---|---|---|---|---|---|
| 28115 | 6 | 13 | 16 | 10 | 8 | 5 | 8 | 13 | 6 |
| 29997 | 6 | 31 | 40 | 22 | 10 | 22 | 20 | 24 | 11 |
| 197927 | | 9 | 11 | 8 | 7 | | 7 | 9 | |
| 221807 | 6 | 16 | 19 | 13 | 16 | 8 | 10 | 13 | 9 |
| 236582 | 6 | 23 | 23 | 23 | 13 | 8 | 16 | 26 | 17 |
| 242745 | 3 | 16 | 22 | 12 | 15 | 9 | 13 | 18 | 13 |
| 357520 | 9 | 25 | 29 | 16 | 7 | 18 | 14 | 21 | 9 |
| 1093240 | 11 | 21 | 22 | 21 | 9 | 13 | 13 | 17 | 14 |
| 1384445 | 10 | 28 | 33 | 23 | 11 | 19 | 19 | 20 | 20 |
| Pepsin C | 7 | 16 | 16 | 19 | 11 | 7 | 12 | 15 | NA |

* highest co-expression marked in bold for each column

VI Transcript Imaging

A transcript image was performed using the LIFESEQ GOLD database (Jun01release, Incyte Genomics). This process allowed assessment of the relative abundance of the expressed cDNAs in more than 1400 cDNA libraries. All sequences and cDNA libraries in the LIFESEQ database have been categorized by system, organ/tissue and cell type. For each category, the number of libraries in which the sequence was expressed were counted and shown over the total number of libraries in that category. In some transcript images, all normalized or subtracted libraries, which have high copy number sequences removed prior to processing, and all mixed or pooled tissues, which are considered non-specific in that they contain more than one tissue type or more than one subject's tissue, can be excluded from the analysis. Treated and untreated cell lines and/or fetal tissue data can also be disregarded or removed where clinical relevance is emphasized. Conversely, fetal tissue may be emphasized wherever elucidation of inherited disorders or differentiation of particular cells or organs from stem cells (such as nerves, heart or kidney) would be furthered by removing clinical samples from the analysis.

For purposes of example, the transcript image for SEQ ID NO:7 is presented below. The first column shows library name; the second column, the number of cDNAs sequenced in that library; the third column, the description of the library; the fourth column, absolute abundance of the transcript in the library; and the fifth column, percentage abundance of the transcript in the library.

Category: Respiratory System (Lung)

| Library* | cDNAs | Description of Prostate Tissue | Abundance | % Abundance |
|---|---|---|---|---|
| LUNGTMT03 | 1949 | lung, mw/adenoCA, 43M | 3 | 0.1539 |
| LUNGNOT34 | 3231 | lung, 12M | 1 | 0.0310 |

*Normalized libraries were not considered in this analysis.

Differential expression of SEQ ID NO:7 in lung matched with adenocarcinoma is 5-fold greater by percent abundance than expression in any other lung tissue including the cytologically normal lung tissue used to obtain mRNAs to construct the LUNGNOT34 library. When used in a lung-specific, diagnostic procedure and compared to normal and diseased standards, SEQ ID NO:7 is diagnostic for adenocarcinoma of the lung. This data confirms the co-expression data produced using GBA (above) and the microarray data presented TABLE 1.

VII Hybridization Technologies and Analyses

The HUMAN GENOME GEM series 1-5 microarrays (Incyte Genomics) contain 45,320 array elements which represent 22,632 annotated clusters and 22,688 unannotated clusters. For the UNIGEM series microarrays (Incyte Genomics), Incyte clones were mapped to non-redundant Unigene clusters (Unigene database (build 46), NCBI; Shuler (1997) J Mol Med 75:694-698), and the 5′ clone with the strongest BLAST alignment (at least 90% identity and 100 bp overlap) was chosen, verified, and used in the construction of the microarray. The UNIGEM V 2.0 microarray (Incyte Genomics) contains 8,502 array elements which represent 8,372 annotated genes and 130 unannotated clusters.

Lung tissue samples used in the microarray analysis were obtained from the Roy Castle International Centre for Lung Cancer Research (RCIC), Liverpool, UK.

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37 C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris-HCl, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning Life Sciences, Acton Mass.) by ultrasound in 0.1% SDS and acetone, etching in 4% hydrofluoric acid (VWR Scientific Products, West Chester Pa.), coating with 0.05% aminopropyl silane (Sigma-Aldrich) in 95% ethanol, and curing in a 110 C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60 C.; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100 C. for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37 C. for 10 min. The labeling reaction is stopped by adding 5 μl of 0.2M EDTA, and probe is purified from unincorporated nucleotides using a PROBEQUANT G-50 microcolumn (APB). The purified probe is heated to 100 C. for five min, snap cooled for two min on ice, and used in membrane-based hybridizations as described below.

Probe Preparation for Polymer Coated Slide Hybridization

Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 yI Cy3 or Cy5 labeling mix, 1 μl RNase inhibitor, 1 μl reverse trans 5 μl 1× yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3,3:1, 1:10, 10:1, 1:25, and 25:1 (w/w). The reaction mixture is mixed and incubated at 37 C. for two hr. The reaction mixture is then incubated for 20 min at 85 C., and probes are purified using two successive CHROMASPIN+TE 30 columns (Clontech). Purified probe is ethanol precipitated by diluting probe to 90 μ¹ in DEPC-treated water, adding 2 Ill lmg/ml glycogen, 60 I¹ 5 M sodium acetate, and 300 μl 100% ethanol. The probe is centrifuged for 20 min at 20,800×g, and the pellet is resuspended in 12 μl resuspension buffer, heated to 65 C. for five min, and mixed thoroughly. The probe is heated and mixed as before and then stored on ice. Probe is used in high density array-based hybridizations as described below.

Membrane-based Hybridization

Membranes are pre-hybridized in hybridization solution containing 1% Sarkosyl and lx high phosphate buffer (0.5 M NaCl, 0.1 M Na₂HPO₄, 5 mM EDTA, pH 7) at 55 C. for two hr. The probe, diluted in 15 ml fresh hybridization solution, is then added to the membrane. The membrane is hybridized with the probe at 55 C. for 16 hr. Following hybridization, the membrane is washed for 15 min at 25 C. in ImM Tris (pH 8.0), 1% Sarkosyl, and four times for 15 min each at 25 C. in lmM Tris (pH 8.0). To detect hybridization complexes, XOMAT-AR film (Eastman Kodak, Rochester N.Y.) is exposed to the membrane overnight at −70 C., developed, and examined visually.

Polymer Coated Slide-based Hybridization

Probe is heated to 65 C. for five min, centrifuged five min at 9400 rpm in a 5415 C. microcentrifuge (Eppendorf Scientific, Westbury N.Y.), and then 18 μl are aliquoted onto the array surface and covered with a coverslip. The arrays are transferred to a waterproof chamber having a cavity just slightly larger than a microscope slide. The chamber is kept at 100% humidity internally by the addition of 140 A¹ of 5×SSC in a corner of the chamber. The chamber containing the arrays is incubated for about 6.5 hr at 60 C. The arrays are washed for 10 min at 45 C. in lxSSC, 0.1% SDS, and three times for 10 min each at 45 C. in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of cDNAs in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to substantially equal numbers of probes derived from both biological samples give a distinct combined fluorescence (Shalon WO95/35505).

Hybridization complexes are detected with a microscope equipped with an INNOVA 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

VIII Complementary Molecules

Molecules complementary to the cDNA, from about 5 bp to about 5000 bp (complement of an entire cDNA insert), are used to detect or inhibit gene expression. These molecules are selected using LASERGENE software (DNASTAR). Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if appropriate elements for inducing vector replication are used in the transformation/expression system.

Stable transformation of appropriate dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

IX Protein Expression

Expression and purification of the protein are achieved using either a cell expression system or an insect cell expression system. The pUB6/V5-His vector system (Invitrogen, Carlsbad Calif.) is used to express protein in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the cDNA by homologous recombination and the polyhedrin promoter drives cDNA transcription. The protein is synthesized as a fusion protein with 6×his which enables purification as described above. Purified protein is used in the following activity and to make antibodies.

X Production of Antibodies

The protein is purified using polyacrylamide gel electrophoresis and used to immunize mice or rabbits. Antibodies are produced using the protocols below. Alternatively, the amino acid sequence of the expressed protein is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using a 431A peptide synthesizer (Applied Biosystems) using Fmoc-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XI Purification of Naturally Occurring Protein Using Specific Antibodies

Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XII Western Analysis

Electrophoresis and Blotting

Samples containing protein are mixed in 2×loading buffer, heated to 95 C. for 3-5 min, and loaded on 4-12% NUPAGE Bis-Tris precast gel (Invitrogen). Unless indicated, equal amounts of total protein are loaded into each well. The gel is electrophoresed in 1×MES or MOPS running buffer (Invitrogen) at 200 V for approximately 45 min on an Xcell II apparatus (Invitrogen) until the RAINBOW marker (APB) resolves, and dye front approaches the bottom of the gel. The gel and its supports are removed from the apparatus and soaked in 1×transfer buffer (Invitrogen) with 10% methanol for a few minutes; and the PVDF membrane is soaked in 100% methanol for a few seconds to activate it. The membrane, the gel, and supports are placed on the TRANSBLOT SD transfer apparatus (Biorad, Hercules Calif.) and a constant current of 350 mAmps is applied for 90 min.

Conjugation with Antibody and Visualization

After the proteins are transferred to the membrane, it is blocked in 5% (w/v) non-fat dry milk in 1× phosphate buffered saline (PBS) with 0.1% Tween 20 detergent (blocking buffer) on a rotary shaker for at least 1 hr at room temperature or at 4 C. overnight. After blocking, the buffer is removed, and 10 ml of primary antibody in blocking buffer is added and incubated on the rotary shaker for 1 hr at room temperature or overnight at 4 C. The membrane is washed 3× for 10 min each with PBS-Tween (PBST), and secondary antibody, conjugated to horseradish peroxidase, is added at a 1:3000 dilution in 10 ml blocking buffer. The membrane and solution are shaken for 30 min at room temperature and then washed three times for 10 min each with PBST.

The wash solution is carefully removed, and the membrane is moistened with ECL+chemiluminescent detection system (APB) and incubated for approximately 5 min. The membrane, protein side down, is placed on BIOMAX M film (Eastman Kodak) and developed for approximately 30 seconds.

XIII Antibody Arrays

Protein:Protein Interactions

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Proteomic Profiles

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt, supra)

XIV Screening Molecules for Specific Binding with the cDNA or Protein

The cDNA, or fragments thereof, or the protein, or portions thereof, are labeled with ³²P-dCTP, Cy3-dCTP, or Cy5-dCTP (APB), or with BIODIPY or FITC (Molecular Probes), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XV Two-Hybrid Screen

A yeast two-hybrid system, MATCHMAKER LexA Two-Hybrid system (Clontech Laboratories), is used to screen for peptides that bind the protein of the invention. A cDNA encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30 C. until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and CDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30 C. A sample of the culture is plated on SD/-Trp/-Ura media and incubated at 30 C. until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the protein, is isolated from the yeast cells and characterized.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| SEQ ID | GEM | Log2(Cy5/Cy3) | Normal Sample (Cy3) | Tumor Sample (Cy5) | Donor |
|---|---|---|---|---|---|
| 1 | HG2 | 1.646363 | mw/NSC AdenoCA | NSC AdenoCA | Dn7964 |
| 1 | HG2 | 1.529918 | mw/SCC | SCC | Dn5792 |
| 1 | HG2 | 1.387926 | Right Lobe, mw/Adenosquamous CA | Right Lobe, Adenosquamous CA | Dn7176 |
| 1 | HG2 | 1.338585 | Left, mw/SCC | Left, SCC | Dn7191 |
| 1 | HG2 | 1.31719 | Left, mw/SCC | Left, SCC | Dn7190 |
| 1 | HG2 | 1.316629 | mw/SCC | SCC | Dn5797 |
| 1 | HG2 | 1.306387 | mw/NSC CA | NSC CA | Dn7973 |
| 1 | HG2 | 1.294447 | Left, mw/SCC | Left, SCC | Dn7196 |
| 1 | HG2 | 1.242977 | mw/SCC | SCC | Dn5793 |
| 1 | HG2 | 1.222392 | Right Upper Lobe, mw/SCC | Right Upper Lobe, SCC | Dn7194 |
| 1 | HG2 | 1.125531 | mw/Squamous NSC CA | Squamous NSC CA | Dn7972 |
| 1 | HG2 | 1.031709 | mw/NSC AdenoCA | NSC AdenoCA | Dn7967 |
| 1 | HG2 | 1.025763 | mw/NSC CA | NSC CA | Dn7963 |
| 2 | HG1 | 6.752705 | mw/NSC AdenoCA | NSC AdenoCA | Dn7964 |
| 2 | HG1 | 4.986508 | mw/NSC AdenoCA | NSC AdenoCA | Dn7964 |
| 2 | HG1 | 6.431647 | mw/NSC CA | NSC CA | Dn7973 |
| 2 | HG1 | 4.423965 | mw/NSC CA | NSC CA | Dn7973 |
| 2 | HG1 | 5.101538 | Left Upper Lobe, mw/AdenoCA | Left Upper Lobe, AdenoCA | Dn7189 |
| 2 | HG1 | 4.907882 | Left Upper Lobe, mw/AdenoCA | Left Upper Lobe, AdenoCA | Dn7189 |
| 2 | HG1 | 5.001461 | Left, mw/SCC | Left, SCC | Dn7190 |
| 2 | HG1 | 4.372349 | Left, mw/SCC | Left, SCC | Dn7190 |
| 2 | HG1 | 4.493963 | Right Upper Lobe, mw/AdenoCA | Right Upper Lobe, AdenoCA | Dn7188 |
| 2 | HG1 | 4.032505 | Right Upper Lobe, mw/AdenoCA | Right Upper Lobe, AdenoCA | Dn7188 |
| 2 | HG1 | 4.470451 | mw/NSC CA | NSC CA | Dn7963 |
| 2 | HG1 | 3.572023 | mw/NSC CA | NSC CA | Dn7963 |
| 2 | HG1 | 4.204642 | mw/NSC AdenoCA | NSC AdenoCA | Dn7965 |
| 2 | HG1 | 3.444601 | mw/NSC AdenoCA | NSC AdenoCA | Dn7965 |
| 2 | HG1 | 4.082362 | Left, mw/SCC | Left, SCC | Dn7191 |
| 2 | HG1 | 3.259982 | Left, mw/SCC | Left, SCC | Dn7191 |
| 2 | HG1 | 3.905454 | Right Upper Lobe, mw/AdenoCA | Right Upper Lobe, AdenoCA | Dn7179 |
| 2 | HG1 | 2.928608 | Right Upper Lobe, mw/AdenoCA | Right Upper Lobe, AdenoCA | Dn7179 |
| 2 | HG1 | 3.61425 | mw/NSC AdenoCA | NSC AdenoCA | Dn7969 |
| 2 | HG1 | 3.161414 | mw/NSC AdenoCA | NSC AdenoCA | Dn7969 |
| 2 | HG1 | 3.4285 | Right Lobe, mw/Adenosquamous CA | Right Lobe, Adenosquamous CA | Dn7176 |
| 2 | HG1 | 3.099295 | Right Lobe, mw/Adenosquamous CA | Right Lobe, Adenosquamous CA | Dn7176 |
| 2 | UG2 | 2.54046 | mw/AdenoCA | AdenoCA | Dn3969 |
| 2 | HG1 | 2.051604 | mw/AdenoCA | AdenoCA | Dn3969 |
| 2 | HG1 | 3.416565 | Right, mw/SCC | Right, SCC | Dn7173 |
| 2 | HG1 | 3.210985 | mw/AdenoCA | AdenoCA | Dn5799 |
| 2 | HG1 | 3.108216 | mw/SCC | SCC | Dn5792 |
| 2 | HG1 | 2.81454 | mw/Large Cell Endocrine CA | Large Cell Endocrine CA | Dn7162 |
| 2 | HG1 | 2.266792 | mw/SCC | SCC | Dn5800 |
| 2 | HG1 | 2.245963 | mw/Carcinoid | Carcinoid | Dn7164 |
| 2 | HG1 | 2.081269 | mw/SCC | SCC | Dn5796 |
| 2 | HG1 | 2.04421 | Left, mw/AdenoCA | Left, AdenoCA | Dn7197 |
| 2 | HG1 | 2.029827 | mw/CA | CA | Dn3837 |
| 2 | HG1 | 1.869842 | mw/SCC | SCC, Dn5797 | Dn5797 |
| 2 | HG1 | 1.757048 | mw/AdenoCA | AdenoCA | Dn5795 |
| 2 | HG1 | 1.649574 | mw/SCC | SCC | Dn5793 |
| 2 | HG1 | 1.586417 | mw/NSC AdenoCA | NSC AdenoCA | Dn7967 |
| 2 | HG1 | 1.46937 | mw/NSC AdenoCA | NSC AdenoCA | Dn7967 |
| 2 | HG1 | 1.365865 | Right Upper Lobe, mw/SCC | Right Upper Lobe, SCC | Dn7194 |
| 2 | HG1 | 1.317392 | Right Upper Lobe, mw/SCC, | Right Upper Lobe, SCC | Dn7194 |
| 2 | HG1 | 1.338802 | Right Upper Lobe, mw/AdenoCA | Right Upper Lobe, AdenoCA | Dn7175 |
| 2 | HG1 | 1.305951 | Left Upper Lobe, mw/SCC | Left Upper Lobe, SCC | Dn7178 |
| 2 | HG1 | 1.244776 | Left, mw/SCC | Left, SCC | Dn7196 |
| 2 | HG1 | 1.17024 | Left, mw/SCC | Left, SCC | Dn7196 |
| 3 | HG2 | 3.745712 | Left, mw/SCC | Left, SCC | Dn7190 |
| 3 | HG2 | 3.613191 | Right Lobe, mw/Adenosquamous CA | Right Lobe, Adenosquamous CA | Dn7176 |
| 3 | HG2 | 3.459432 | mw/NSC CA | NSC CA | Dn7963 |
| 3 | HG2 | 3.375426 | mw/NSC CA | NSC CA | Dn7963 |
| 3 | HG2 | 3.360469 | mw/NSC AdenoCA | NSC AdenoCA | Dn7964 |
| 3 | HG2 | 3.284345 | mw/NSC AdenoCA | NSC AdenoCA | Dn7964 |
| 3 | HG2 | 3.089298 | mw/SCC | SCC | Dn5792 |
| 3 | HG2 | 2.873697 | Right Upper Lobe, mw/SCC | Right Upper Lobe, SCC | Dn7194 |
| 3 | HG2 | 2.809806 | Left, mw/SCC | Left, SCC | Dn7191 |
| 3 | HG2 | 2.676488 | mw/SCC | SCC | Dn5797 |
| 3 | HG2 | 2.665422 | mw/SCC | SCC | Dn5796 |
| 3 | HG2 | 2.5243 | mw/NSC AdenoCA | NSC AdenoCA | Dn7969 |
| 3 | HG2 | 1.955454 | mw/NSC AdenoCA | NSC AdenoCA | Dn7969 |
| 3 | HG2 | 2.522255 | Left, mw/SCC | Left, SCC | Dn7196 |
| 3 | HG2 | 2.471464 | mw/Large Cell Endocrine CA | Large Cell Endocrine CA | Dn7162 |
| 3 | HG2 | 2.417235 | mw/NSC CA | NSC CA | Dn7973 |
| 3 | HG2 | 2.353637 | mw/NSC CA | NSC CA | Dn7973 |
| 3 | HG2 | 2.377361 | mw/SCC | SCC | Dn5793 |
| 3 | HG2 | 2.36257 | Left Upper Lobe, mw/AdenoCA | Left Upper Lobe, AdenoCA | Dn7189 |
| 3 | HG2 | 2.276308 | mw/AdenoCA | AdenoCA | Dn5799 |
| 3 | HG2 | 2.207296 | Right Upper Lobe, mw/AdenoCA | Right Upper Lobe, AdenoCA | Dn7188 |
| 3 | HG2 | 2.184167 | Left Upper Lobe, mw/SCC | Left Upper Lobe, SCC | Dn7178 |
| 3 | HG2 | 2.063242 | Right Upper Lobe, mw/AdenoCA | Right Upper Lobe, AdenoCA | Dn7179 |
| 3 | HG2 | 2.001148 | mw/NSC CA | NSC CA | Dn7971 |
| 3 | HG2 | 1.697896 | mw/NSC CA | NSC CA | Dn7971 |
| 3 | HG2 | 1.843196 | mw/NSC AdenoCA | NSC AdenoCA | Dn7967 |
| 3 | HG2 | 1.767682 | mw/NSC AdenoCA | NSC AdenoCA | Dn7967 |
| 3 | HG2 | 1.74116 | mw/SCC | SCC | Dn7168 |
| 3 | HG2 | 1.727418 | mw/SCC | SCC | Dn5800 |
| 3 | HG2 | 1.626439 | Right Upper Lobe, mw/AdenoCA | Right Upper Lobe, AdenoCA | Dn7175 |
| 3 | HG2 | 1.424688 | mw/NSC AdenoCA | NSC AdenoCA | Dn7965 |
| 3 | HG2 | 1.36352 | mw/NSC AdenoCA | NSC AdenoCA | Dn7965 |
| 3 | HG2 | 1.264615 | mw/NSC AdenoCA | NSC AdenoCA | Dn7962 |
| 3 | HG2 | 1.12136 | mw/NSC AdenoCA | NSC AdenoCA | Dn7966 |
| 4 | HG1 | 4.46471 | mw/NSC AdenoCA | NSC AdenoCA | Dn7965 |
| 4 | HG1 | 4.070798 | mw/NSC AdenoCA | NSC AdenoCA | Dn7965 |
| 4 | HG1 | 3.52097 | mw/SCC | SCC | Dn5792 |
| 4 | HG1 | 3.2488 | Left Upper Lobe, mw/AdenoCA | Left Upper Lobe, AdenoCA | Dn7189 |
| 4 | HG1 | 3.221679 | Left Upper Lobe, mw/AdenoCA | Left Upper Lobe, AdenoCA | Dn7189 |
| 4 | HG1 | 3.178487 | Right, mw/SCC | Right, SCC | Dn7173 |
| 4 | HG1 | 3.156635 | Right Upper Lobe, mw/AdenoCA | Right Upper Lobe, AdenoCA | Dn7188 |
| 4 | HG1 | 2.876319 | Right Upper Lobe, mw/AdenoCA | Right Upper Lobe, AdenoCA | Dn7188 |
| 4 | HG1 | 2.816926 | mw/NSC CA | NSC CA | Dn7973 |
| 4 | HG1 | 2.805592 | mw/NSC AdenoCA | NSC AdenoCA | Dn7969 |
| 4 | HG1 | 2.403921 | mw/NSC AdenoCA | NSC AdenoCA | Dn7969 |
| 4 | HG1 | 2.68729 | Left, mw/AdenoCA | Left, AdenoCA | Dn7197 |
| 4 | HG1 | 2.595121 | mw/SCC | SCC | Dn5800 |
| 4 | HG1 | 2.510888 | mw/NSC CA | NSC CA | Dn7963 |
| 4 | HG1 | 1.767456 | mw/NSC CA | NSC CA | Dn7963 |
| 4 | HG1 | 2.473999 | mw/Carcinoid | Carcinoid | Dn7164 |
| 4 | HG1 | 2.434125 | mw/Large Cell Endocrine CA | Large Cell Endocrine CA | Dn7162 |
| 4 | HG1 | 2.399958 | mw/SCC | SCC | Dn5796 |
| 4 | HG1 | 2.280555 | mw/NSC AdenoCA | NSC AdenoCA | Dn7964 |
| 4 | HG1 | 2.170923 | mw/NSC AdenoCA | NSC AdenoCA | Dn7964 |
| 4 | HG1 | 2.13245 | mw/NSC CA | NSC CA | Dn7973 |
| 4 | HG1 | 2.068865 | Left Upper Lobe, mw/SCC | Left Upper Lobe, SCC | Dn7178 |
| 4 | HG1 | 1.756633 | Left Upper Lobe, mw/SCC | Left Upper Lobe, SCC | Dn7178 |
| 4 | HG1 | 1.956383 | mw/SCC | SCC | Dn5797 |
| 4 | HG1 | 1.878231 | Left, mw/SCC | Left, SCC | Dn7191 |
| 4 | HG1 | 1.647815 | Left, mw/SCC | Left, SCC | Dn7191 |
| 4 | HG1 | 1.785894 | Right Upper Lobe, mw/SCC | Right Upper Lobe, SCC | Dn7194 |
| 4 | HG1 | 1.654822 | Right Upper Lobe, mw/SCC | Right Upper Lobe, SCC | Dn7194 |
| 4 | HG1 | 1.573583 | Right Upper Lobe, mw/AdenoCA | Right Upper Lobe, AdenoCA | Dn7179 |
| 4 | HG1 | 1.451517 | Right Upper Lobe, mw/AdenoCA | Right Upper Lobe, AdenoCA | Dn7179 |
| 4 | HG1 | 1.495027 | Left, mw/SCC | Left, SCC | Dn7190 |
| 4 | HG1 | 1.153306 | Left, mw/SCC | Left, SCC | Dn7190 |
| 7 | HG1 | 5.447411 | Left, mw/SCC | Left, SCC | Dn7191 |
| 7 | HG1 | 4.78807 | Left, mw/SCC | Left, SCC | Dn7191 |
| 7 | HG1 | 4.995174 | Left, mw/SCC | Left, SCC | Dn7190 |
| 7 | HG1 | 4.706784 | Left, mw/SCC | Left, SCC | Dn7190 |
| 7 | HG1 | 4.991896 | mw/NSC AdenoCA | NSC AdenoCA | Dn7965 |
| 7 | HG1 | 4.425823 | mw/NSC AdenoCA | NSC AdenoCA | Dn7965 |
| 7 | HG1 | 4.749855 | Right Lobe, mw/Adenosquamous CA | Right Lobe, Adenosquamous CA | Dn7176 |
| 7 | HG1 | 4.336989 | Right Lobe, mw/Adenosquamous CA | Right Lobe, Adenosquamous CA | Dn7176 |
| 7 | HG1 | 4.287545 | Left Upper Lobe, mw/AdenoCA | Left Upper Lobe, AdenoCA | Dn7189 |
| 7 | HG1 | 3.774911 | Left Upper Lobe, mw/AdenoCA | Left Upper Lobe, AdenoCA | Dn7189 |
| 7 | HG1 | 4.218006 | Right, mw/SCC | Right, SCC | Dn7173 |
| 7 | HG1 | 4.0318 | Right Upper Lobe, mw/AdenoCA | Right Upper Lobe, AdenoCA | Dn7179 |
| 7 | HG1 | 3.826022 | Right Upper Lobe, mw/AdenoCA | Right Upper Lobe, AdenoCA | Dn7179 |
| 7 | HG1 | 3.54305 | Left Upper Lobe, mw/SCC | Left Upper Lobe, SCC | Dn7178 |
| 7 | HG1 | 2.962044 | Left Upper Lobe, mw/SCC | Left Upper Lobe, SCC | Dn7178 |
| 7 | HG1 | 3.448008 | mw/NSC AdenoCA | NSC AdenoCA | Dn7969 |
| 7 | HG1 | 3.377626 | mw/NSC AdenoCA | NSC AdenoCA | Dn7969 |
| 7 | HG1 | 3.380674 | mw/SCC | SCC | Dn5800 |
| 7 | HG1 | 3.315486 | mw/SCC | SCC | Dn5792 |
| 7 | HG1 | 3.262878 | mw/SCC | SCC | Dn5797 |
| 7 | HG1 | 3.138934 | mw/Carcinoid | Carcinoid | Dn7164 |
| 7 | HG1 | 3.009344 | Right Upper Lobe, mw/SCC | Right Upper Lobe, SCC | Dn7194 |
| 7 | HG1 | 2.675871 | Right Upper Lobe, mw/SCC | Right Upper Lobe, SCC | Dn7194 |
| 7 | HG1 | 3 | mw/NSC CA | NSC CA | Dn7963 |
| 7 | HG1 | 2.999329 | mw/NSC CA | NSC CA | Dn7963 |
| 7 | HG1 | 2.914621 | mw/SCC | SCC | Dn5796 |
| 7 | HG1 | 2.866545 | mw/AdenoCA | AdenoCA | Dn5799 |
| 7 | HG1 | 2.811725 | mw/Large Cell Endocrine CA | Large Cell Endocrine CA | Dn7162 |
| 7 | HG1 | 2.680182 | Left, mw/SCC | Left, SCC | Dn7196 |
| 7 | HG1 | 2.34163 | Left, mw/SCC | Left, SCC | Dn7196 |
| 7 | HG1 | 2.485831 | mw/SCC | SCC | Dn5793 |
| 7 | HG1 | 2.387317 | Right Upper Lobe, mw/AdenoCA | Right Upper Lobe, AdenoCA | Dn7188 |
| 7 | HG1 | 2.08713 | Right Upper Lobe, mw/AdenoCA | Right Upper Lobe, AdenoCA | Dn7188 |
| 7 | HG1 | 2.296617 | mw/NSC AdenoCA | NSC AdenoCA | Dn7964 |
| 7 | HG1 | 2.189015 | mw/NSC AdenoCA | NSC AdenoCA | Dn7964 |
| 7 | HG1 | 1.694965 | Left, mw/AdenoCA | Left, AdenoCA | Dn7197 |
| 7 | HG1 | 1.484006 | mw/NSC CA | NSC CA | Dn7973 |
| 7 | HG1 | 1.262141 | mw/NSC CA | NSC CA | Dn7973 |
| 7 | HG1 | 1.337452 | mw/SCC | SCC | Dn7168 |
| 7 | HG1 | 1.245464 | mw/NSC AdenoCA, | NSC AdenoCA | Dn7967 |
| 7 | HG1 | 1.011795 | mw/NSC AdenoCA, | NSC AdenoCA | Dn7967 |
| 8 | HG5 | 2.511884 | Left, mw/SCC | Left, SCC | Dn7190 |
| 8 | HG5 | 2.406208 | Left, mw/AdenoCA | Left, AdenoCA | Dn7197 |
| 8 | HG5 | 2.398364 | Right Lobe, mw/Adenosquamous CA | Right Lobe, Adenosquamous CA | Dn7176 |
| 8 | HG5 | 2.128511 | Right Lobe, mw/Adenosquamous CA | Right Lobe, Adenosquamous CA | Dn7176 |
| 8 | HG5 | 2.389336 | mw/NSC AdenoCA | NSC AdenoCA | Dn7964 |
| 8 | HG5 | 1.918563 | mw/NSC AdenoCA | NSC AdenoCA | Dn7964 |
| 8 | HG5 | 2.299936 | Right Upper Lobe, mw/AdenoCA | Right Upper Lobe, AdenoCA | Dn7188 |
| 8 | HG5 | 1.744967 | Right Upper Lobe, mw/AdenoCA | Right Upper Lobe, AdenoCA | Dn7188 |
| 8 | HG5 | 2.25621 | mw/NSC AdenoCA | NSC AdenoCA | Dn7969 |
| 8 | HG5 | 1.593983 | mw/NSC AdenoCA | NSC AdenoCA | Dn7969 |
| 8 | HG5 | 2.229215 | Right Upper Lobe, mw/SCC | Right Upper Lobe, SCC | Dn7194 |
| 8 | HG5 | 1.351635 | Right Upper Lobe, mw/SCC | Right Upper Lobe, SCC | Dn7194 |
| 8 | HG5 | 2.050715 | mw/NSC AdenoCA | NSC AdenoCA | Dn7965 |
| 8 | HG5 | 1.374661 | mw/NSC AdenoCA | NSC AdenoCA | Dn7965 |
| 8 | HG5 | 2.019549 | Left, mw/SCC | Left, SCC | Dn7196 |
| 8 | HG5 | 1.849666 | Left, mw/SCC | Left, SCC | Dn7196 |
| 8 | HG5 | 1.988833 | mw/SCC | SCC | Dn5792 |
| 8 | HG5 | 1.93308 | mw/NSC CA | NSC CA | Dn7973 |
| 8 | HG5 | 1.187323 | mw/NSC CA | NSC CA | Dn7973 |
| 8 | HG5 | 1.910243 | Left Upper Lobe, mw/AdenoCA | Left Upper Lobe, AdenoCA | Dn7189 |
| 8 | HG5 | 1.702125 | Left Upper Lobe, mw/AdenoCA | Left Upper Lobe, AdenoCA | Dn7189 |
| 8 | HG5 | 1.838499 | Right, mw/SCC | Right, SCC | Dn7173 |
| 8 | HG5 | 1.789628 | mw/AdenoCA | AdenoCA | Dn5799 |
| 8 | HG5 | 1.759771 | mw/NSC AdenoCA | NSC AdenoCA | Dn7967 |
| 8 | HG5 | 1.490854 | mw/NSC AdenoCA | NSC AdenoCA | Dn7967 |
| 8 | HG5 | 1.56939 | mw/NSC CA | NSC CA | Dn7963 |
| 8 | HG5 | 1.555389 | Right Upper Lobe, mw/AdenoCA | Right Upper Lobe, AdenoCA | Dn7179 |
| 8 | HG5 | 1.515849 | Left Upper Lobe, mw/SCC | Left Upper Lobe, SCC | Dn7178 |
| 8 | HG5 | 1.484732 | mw/Large Cell Endocrine CA | Large Cell Endocrine CA | Dn7162 |
| 8 | HG5 | 1.360429 | mw/SCC | SCC | Dn5800 |
| 8 | HG5 | 1.158196 | Left, mw/SCC | Left, SCC | Dn7191 |
| 8 | HG5 | 1.121402 | mw/Carcinoid | Carcinoid | Dn7164 |
| 8 | HG5 | 1.114478 | mw/SCC | SCC | Dn5796 |
| 8 | HG5 | 1.015616 | mw/SCC | SCC | Dn5793 |
| 8 | HG5 | 1.010788 | Right Upper Lobe, mw/AdenoCA | Right Upper Lobe, AdenoCA | Dn7175 |
| 9 | HG5 | 5.639039 | Left, mw/SCC | Left, SCC | Dn7191 |
| 9 | HG5 | 4.139617 | mw/NSC CA | NSC CA | Dn7963 |
| 9 | HG5 | 3.200801 | Right Upper Lobe, mw/SCC | Right Upper Lobe, SCC | Dn7194 |
| 9 | HG5 | 2.767323 | Right Upper Lobe, mw/SCC | Right Upper Lobe, SCC | Dn7194 |
| 9 | HG5 | 2.70044 | Left, mw/SCC | Left, SCC | Dn7196 |
| 9 | HG5 | 1.988351 | Left, mw/SCC | Left, SCC | Dn7196 |
| 9 | HG5 | 2.57289 | mw/NSC AdenoCA | NSC AdenoCA | Dn7967 |
| 9 | HG5 | 1.234982 | mw/NSC AdenoCA | NSC AdenoCA | Dn7967 |
| 9 | HG5 | 2.512251 | Right Upper Lobe, mw/AdenoCA | Right Upper Lobe, AdenoCA | Dn7175 |
| 9 | HG5 | 1.453032 | Right Upper Lobe, mw/AdenoCA | Right Upper Lobe, AdenoCA | Dn7175 |
| 9 | HG5 | 2.238565 | mw/NSC CA | NSC CA | Dn7973 |
| 9 | HG5 | 1.099615 | mw/NSC CA | NSC CA | Dn7973 |
| 9 | HG5 | 2.19137 | mw/Carcinoid | Carcinoid | Dn7164 |
| 9 | HG5 | 2.094227 | Left, mw/AdenoCA | Left, AdenoCA | Dn7197 |
| 9 | HG5 | 2.044479 | Left, mw/SCC | Left, SCC | Dn7190 |
| 9 | HG5 | 2.016278 | mw/NSC AdenoCA | NSC AdenoCA | Dn7964 |
| 9 | HG5 | 1.476861 | mw/NSC AdenoCA | NSC AdenoCA | Dn7964 |
| 9 | HG5 | 1.870774 | Right Upper Lobe, mw/AdenoCA | Right Upper Lobe, AdenoCA | Dn7188 |
| 9 | HG5 | 1.810273 | Right Upper Lobe, mw/AdenoCA | Right Upper Lobe, AdenoCA | Dn7188 |
| 9 | HG5 | 1.749524 | Right, mw/SCC | Right, SCC | Dn7173 |
| 9 | HG5 | 1.742649 | mw/NSC AdenoCA | NSC AdenoCA | Dn7962 |
| 9 | HG5 | 1.651037 | Right Lobe, mw/Adenosquamous CA | Right Lobe, Adenosquamous CA | Dn7176 |
| 9 | HG5 | 1.508217 | Right Lobe, mw/Adenosquamous CA | Right Lobe, Adenosquamous CA | Dn7176 |
| 9 | HG5 | 1.528386 | mw/Large Cell Endocrine CA | Large Cell Endocrine CA | Dn7162 |
| 9 | HG5 | 1.409099 | Left Upper Lobe, mw/AdenoCA | Left Upper Lobe, AdenoCA | Dn7189 |
| 9 | HG5 | 1.017656 | Left Upper Lobe, mw/AdenoCA | Left Upper Lobe, AdenoCA | Dn7189 |
| 9 | HG5 | 1.388172 | mw/SCC | SCC | Dn5797 |
| 9 | HG5 | 1.279073 | mw/NSC CA | NSC CA | Dn7971 |
| 9 | HG5 | 1.098599 | Left Upper Lobe, mw/SCC | Left Upper Lobe, SCC | Dn7178 |
| 9 | HG5 | 1.012085 | mw/NSC AdenoCA | NSC AdenoCA | Dn7969 |
| pepsin C | UG2 | 2.577476 | mw/AdenoCA | AdenoCA | Dn3969 |

What is claimed is:

1. A combination comprising a plurality of cDNAs having the nucleic acid sequences of SEQ ID NOs:1-9 and a cDNA encoding pepsin C and the complements of these cDNAs.

2. An isolated cDNA comprising a nucleic acid sequence selected from SEQ ID NOs:1-9 and the complements thereof.

3. A method for using a combination comprising a plurality of cDNAs to detect expression of a nucleic acid in a sample comprising:
a) hybridizing the combination of claim 1 to nucleic acids of the sample under conditions to form at least one hybridization complex; and
b) detecting hybridization complex formation, wherein complex formation indicates expression of the nucleic acid in the sample.

4. The method of claim 3 further comprising amplifying the nucleic acids of the sample prior to hybridization.

5. The method of claim 3 wherein the sample is from lung.

6. The method of claim 3 wherein complex formation is compared to standards and indicates differential expression.

7. The method of claim 6 wherein differential expression is diagnostic of a lung disorder.

8 A substrate upon which the combination of claim 1 is arrayed.

9. A method of using a combination to screen a plurality of molecules to identify at least one ligand which specifically binds a cDNA of the combination, the method comprising:
a) contacting the substrate of claim 8 with molecules under conditions to allow specific binding; and
b) detecting specific binding, thereby identifying a ligand which specifically binds a cDNA of the combination.

10. The method of claim 9 wherein the molecules to be screened and purified are selected from DNA molecules, RNA molecules, peptides, and proteins.

11. A composition comprising a cDNA of claim 2 and a labeling moiety.

12. A vector comprising a cDNA of claim 2.

13. A host cell comprising the vector of claim 12.

14. A method for using a host cell to produce a protein, the method comprising:
a) culturing the host cell of claim 13 under conditions for expression of the protein; and
b) recovering the protein from cell culture.

15. A purified protein obtained using the method of claim 14.

16. A composition comprising the protein of claim 15 and a pharmaceutical carrier.

17. A method for using a protein to screen a plurality of molecules to identify at least one ligand which specifically binds the protein, the method comprising:
a) combining the protein of claim 15 with the plurality of molecules under conditions to allow specific binding; and
b) detecting specific binding, thereby identifying a ligand which specifically binds the protein.

18. The method of claim 17 wherein the plurality of molecules is selected from antibodies, DNA molecules, RNA molecules, proteins, and small drug molecules.

19. A method of using a protein to prepare and purify an antibody comprising:
a) immunizing an animal with the protein of claim 15 under conditions to elicit an antibody response;
b) isolating animal antibodies;
c) attaching the protein to a substrate;
d) contacting the substrate with isolated antibodies under conditions to allow specific binding to the protein;
e) dissociating antibody from the protein, thereby obtaining purified antibody.

20. An antibody produced by the method of claim 19.
